(19) United States
(12) Patent Application Publication
(10) Pub. No.: US 2004/0077046 A1
(43) Pub. Date: Apr. 22, 2004

(54) Histone deacetylase-related gene and protein

(76) Inventors: Dalia Cohen, Livingston, NJ (US); Umesh Bhatia, San Jose, CA (US); Richard Lie Cai, Bridgewater, NJ (US); Denise Dawn Fischer, Bernardsville, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/451,316

(22) PCT Filed: Dec. 18, 2001
(86) PCT No.: PCT/EP01/14928

(30) Foreign Application Priority Data
Dec. 20, 2000 (US) 60256827
May 23, 2001 (US) 60293089
Sep. 6, 2001 (US) 60317984

(51) Int. Cl. C07H 21/04; C12N 9/16; C07K 14/47

(52) U.S. Cl. 435/69.1; 435/320.1; 435/196; 435/325; 530/350; 536/23.2

(57) ABSTRACT

Disclosed is an HDAC related genes and gene products. In particular, the invention relates to a protein and variants that is highly homologous to known HDACs and referred to herein as HDAC9, nucleic acid molecules that encode such a protein, antobodies that recognize the protein, and methods for diagnosing conditions related to abnormal HDAC9 activity or gene expression.

### FIELD OF THE INVENTION

This invention relates to a histone deacetylase gene and gene product. In particular, the invention relates to a protein that is highly homologous to known yeast histone deacetylase 1 pedal) class II histone deacetylases (HDACs), nucleic acid molecules that encode such a protein, antibodies that recognize the protein, and methods for diagnosing conditions related to abnormal HDAC activity, including, for example, abnormal cell proliferation, cancer, atherosclerosis, inflammatory bowel disease, host inflammatory or immune response or psoriasis.

### BACKGROUND OF THE INVENTION

Histone acetylation is a major regulatory mechanism that modulates gene expression by altering the accessibility of transcription factors to DNA. Acetylation of histones is a reversible modification of the free Σ-amino group of lysine that occurs during the assembly of nucleosomes and during DNA synthesis. Changes in histone acetylation levels also occur during transcriptional activation and silencing. Acetylation of histones is generally associated with transcriptional activity, whereas deacetylation is associated with transcriptional repression. Histone acetylation levels result from an equilibrium between competing histone acetylases and deacetylases (Emiliani, S., Fischle, W., Van Lindt, C., Al-Abed, Y., and Verdin, E., Proc Nat. Acad. Sci., U.S.A., 95, 2795-2800 (1998).

HDACs have been shown to play an important role in the regulation of transcription. HDACs function as components of complexes that are involved in transcriptional repression. This is mediated through interactions of HDACs with multi-protein complexes and requires deacetylase activity. HDAC complexes may contain the co-repressor mSin3A (Kasten, M. M., Dorland, S., Stillman, D. J. Mol. Cell. Biol. 17, 4852-4858 (1997)) and mSin3A-associated proteins (Zhang, Y., Iratni, R., Erdjument-Bromage, H., Tempst, P., Reinberg, D. Cell 89, 357-364 (1997); Zhang, Y., Sun, Z. W., Iratni, R., Erdjument-Bromage, H., Tempst, P., Hampsey, M., Reinberg, D. Mol. Cell. 1, 1021-1031(1998)) silencing mediators NcoR (Nagy, L., H.- Y. Kao, D. Chakravarti, R. J. Lin, C. A. Hassig, D. E. Ayer, S. L. Schreiber, and R. M. Evans (1997) Cell 89, 373-380 and SMRT (Alland, L. et al., Nature 387:49-55 (1997); Heinzel, T. et al., Nature 387:43-8 (1997)), transcriptional repressors Rb (Brownell, J. E., Zhou, J., Ranalli, T., Kobayashi, R., Edmondson, D. G., Roth, S. Y., and Allis, C. D. (1996) Cell 84, 843-851), Rb-like proteins p107 (Ferreira, R., Magnaghi-Jaulin, L., Robin, P., Harel-Bellan, A., Trouche, D. (1998) Proc. Natl. Acad. Sci. USA 95, 10493-10498) and p130 (Stiegler, P., De Luca, A. Bagella, L., Giordano, A. (1998) Cancer Res. 389, 187-190), Rb-associated proteins (Nicolas, E., Morales, V., Magnaghi-Jaulin, L., Harel-Bellan, A., Richard-Foy, H., Trouche, D. (2000) J. Biol. Chem. 275, 9797-9804, Lai, A., Lee, J. M., Yang, W. M., DeCaprio, J. A., Kaelin, W. G. Jr., Seto, E., Branton, P. E. (1999) Mol. Cell. Biol. 19, 6632-6641), Mad/Max (Laherty, C., W.- M. Yang, J.- M. Sun, J. R. Davie, E. Seto, and R. N. Eisernan. (1997) Cell 89, 349-456), nuclear hormone receptors (Nagy, L., H.- Y. Kao, D. Chakravarti, R. J. Lin, C. A. Hassig, D. E. Ayer, S. L. Schreiber, and R. M. Evans. (1997) Cell 89, 373-380), nucleosome remodeling factors (Xue, Y., Wong, J., Moreno, G. T., Young, M. K., Cote, J., Wang, W. (1998) Mol. Cell. 2, 851-861), methyl-binding proteins (Fuks, F., Burgers, W. A., Brehm, A., Hughes-Davies, L., Kouzarides, T. (2000) Nat. Genet. 24, 88-91, Nan, X., Ng, H. H., Johnson, C. A., Laherty C. D., Turner, B. M., Eisenman, R. N., Bird, A. (1998) Nature 393, 386-389, Ghosh, A. K., Steele, R., Ray, R. B. (1999) Biochem. Biophys. Res. Commun. 260, 405-409, Ng, H. H., Zhang, Y., Hendrich, B., Johnson, C. A., Turner, B. M., Erdjument-Bromage, H., Tempst, P., Reinberg, D., Bird, A. (1999) Nat. Genet. 23, 58-61), and DNA repair machinery proteins (Yarden, R. I., Brody, L. C. (1999) Proc. Natl. Acad. Sci. U.S.A. 96, 4983-4988, Cai, R. L., Yan-Neale, Y., Cueto, M. A., Xu, H., Cohen, D. (2000) J. Biol. Chem. 275, 27909-27916). Furthermore, HDAC1 has been found to bind directly to YY1 (Yang, W.- M., Inouye, C., Zeng, Y., Bearss, D., and Seto, E. (1996) Proc. Natl. Acad. Sci. 93, 122845-12850) and Sp1 (Doetzlhofer, A., Rotheneder, H., Lagger, G., Koranda, M., Kurtev, V., Brosch, G., Wintersberger, E., Seiser, C. (1999) Mol. Cell. Biol. 19, 5504-5511) and HDACs 4 and 5 bind to MEF2 (Grozinger, C. M., and Schreiber, S. L. (2000) Proc. Natl. Acad. Sci. 97, 7835-7840). In addition, HDACs have been found together in complexes (Eilers, A. L., Billin, A. N., Liu, J., Ayer, D. E. (1999) J Biol Chem 274, 32750-32756, Grozinger, C. M., and Schreiber, S. L. (2000) Proc. Natl. Acad. Sci. 97, 7835-7840).

Two distinct classes of yeast histone deacetylases have been identified based upon size and sequence. Yeast class I HDACs include Rpd3, Hos1p, and Hos2p. Class II contains yeast HDA1p. Furthermore, members of these two classes were found to form different complexes. Human HDACs have been classified based upon their similarity to yeast sequences. Class I human HDACs include HDACs1-3 and 8. Class II HDACs include HDACs 4-7. The deacetylase core of class I HDACs reside in the first ˜390 amino acids. Class II HDAC catalytic domains are located in the C-terminal of these peptides, with the exception of HDAC4 that contains a second catalytic domain in the N-terminus (Grozinger, C. M., Hassig, C. A., and Schreiber, S. L. (1999) Proc. Natl. Acad. Sci. U.S.A. 96, 4868-4873).

An important approach that has been used to study the function of chromatin acetylation is the use of specific inhibitors of histone deacetylase. Several classes of compounds have been identified that inhibit HDAC. Histone deacetylase inhibitors have been found to have anti-proliferative effects, including induction of G1/S and G2/M cell cycle arrest, differentiation (Itazaki, H., K. Nagashima, K Sugita, H. Yoshida, Y. Kawamura, Y. Yasuda, K Matsumoto, K. Ishii, N. Uotani, H. Nakai, A. Terui, S. Yoshimatsu, Y. Ikenishi and Y. Nakagawa. (1990) J. Antibiot. 12, 1524-1532, Hoshikawa, Y., Kijima, M., Yoshida, M., and Beppu, T. (1991) Agric. Biol. Chem. 55, 1491-1497, Hoshikawa, Y., Kwon, H.- J., Yoshida, M., Horinouchi, S., and Beppu, T. (1994) Exp. Cell Res. 214, 189-197, Sugita, K, Koizumi, K., and Yoshida, H. (1992) Cancer Res. 52, 168-172, Yoshida, M., Y. Hoshikawa, K. Koseki, K. Mori and T. Beppu. (1990) J. of Antibiot. 43, 1101-106, Yoshida, M., Nomura, S., and Beppu, T. (1987) Cancer Res. 47, 3688-3691), and apoptosis (Medina, V., Edmonds, B., Young, G. P., James, R., Appleton, S., Zalewski, P. D. (1997) Cancer Res. 57, 3697-3707) of transformed and normal cells and reversal of transformation (Kwon, H. J., Owa, T., Hassig, C. A., Shimada, J., and Schreiber, S. (1998) Proc. Natl. Acad. Sci. U.S.A. 95, 3356-3361, Kim, M. -S., Son, M. -W., Park, Y. I., and Moon, A. (2000) Cancer Lett. 157, 23-30). These effects, along with the presence of HDAC in complexes with fusions of unliganded retinoic acid receptors PML-RARα and PLZF-RARα indicate a role for HDACs in tumorigenicity (Grignani, F., De Matteis, S., Nervi, C., Tomassoni, L., Gelmetti, V., Cioce, M., Fanelli, K, Ruthardt, M., Ferrara, F. F., Zamir, I., Seiser, C., Grignani, F., Lazar, M. A., Minucci, S., Pelicci, P. G. (1998) Nature 391, 815-818, He, L. Z., Guidez, F., Tribioli, C., Peruzzi, D., Ruthardt, M., Zelent, A., Pandolfi, P. P. (1998) Nat. Genet., 18, 126-35, Lin, R. J., Nagy, L., Inoue, S., Shao, W., Miller, W. H. Jr and Evans, R. M. (1998) Nature 391, 811-814). Furthermore, histone deacetylase inhibitors, phenylbutyrate and trichostatin A have shown promise in the treatment of promyelocytic leukemia and several other HDAC inhibitors are being studied and are nearing the clinic (Byrd, J. C., Shinn, C., Ravi, R., Willis, C. R., Waselenko, J. K., Flinn, I. W., Dawson, N. A., Grever, M. R. (1999) Blood 94, 1401-1408, Kim, Y. B., Lee, K. H., Sugita, K., Yoshida, M., Horinouchi, S. (1999) Oncogene 18, 2461-2470, Cohen, L. A., Amin, S., Marks, P. A., Rifkind, R. A., Desai, D., Richon, V. M. (1999) Anticancer Res. 19, 4999-5005). In addition, the HDAC inhibitor, butyrate was found to decrease expression of pro-inflammatory cytokines TNF-α, TNF-β, IL-6, and IL1-β. These effects are thought to result from inhibition of NFKB activation (Segain J P, Raingeard de la Bletiere D, Bourreille, A., Leray V., Gervois, N., Rosales, C., Ferrier, L., Bonnet, C., Blottiere, H. M., Galmiche, J. P. (2000) Butyrate inhibits inflammatory responses through NFkappaB inhibition: implications for Crohn's disease. Gut 47, 397-403) and its ability to inhibit histone deacetylases (Inan M. S., Rasoulpour, R. J., Yin, L., Hubbard, A. K, Rosenberg, D. W., Giardina, C. (2000). The luminal short-chain fatty acid butyrate modulates NF-kappaB activity in a human colonic epithelial cell line. Gastroenterology 118, 724-34).

The discovery of the HDAC inhibitor trapoxin, made it possible to isolate the first human histone deacetylase, HDAC1, using an affinity matrix column to which a trapoxin-like molecule was bound (Taunton, J., Collins, J. L., and Schreiber, S. (1996) J. Am. Chem. Soc. 118, 10412-10422). Subsequently, seven other human HDAC enzyme isoforms were reported (Taunton, J., Hassig, C. A. and Schreiber, S. L. (1996). Science 272, 408-411, Yang, W. m., Inouye, C., Zeng, Y., Bearss, D., and Seto, D. (1996) Proc. Natl. Acad. Sci. U.S.A. 93, 12845-12850, Yang, W. M., Yao, y. L., Sun, J. M., Davie, J. R., and Seto, E. (1997). J. Biol Chem. 272, 28001-28007, Emiliani, S., Fischle, W., Van Lint, C., Al-Abed, Y., and Verdin, E. (1998). Proc. Natl. Acad. Sci. U.S.A. 95, 2795-27800). These 8 HDACs have been divided into class I ( HDACs 1-3 and 8 similar to the yeast gene Rpd3) and class II HDACs (4-7 similar to yeast gene hda1 (Grozinger, C. M., Hassig, C. A., and Schrieber, S. L. (1999). Proc. Natl. Acad. Sci. U.S.A. 96, 4983-4988.) based on sequence homology. Here we report the isolation and characterization of a potential new HDAC, referred to herein as HDAC9, which displays sequence similarity to the hda1 class II HDACs. HDAC9 has characteristics that bridge HDAC class I and class II.

### SUMMARY OF THE INVENTION

The present invention relates to histone deacetylases, in particular to a novel histone deacetylase HDAC9.

In a first aspect, the invention provides an isolated polypeptide comprising an amino acid sequence as set forth in SEQ ID NO:1, SEQ ID NO 5 or SEQ ID NO 6 . Furthermore, the invention provides an isolated polypeptide consisting of an amino acid sequence as set forth in SEQ ID NO:1, SEQ ID NO 5 or SEQ ID NO 6. The amino acid sequence as set forth in SEQ ID NO:1,SEQ ID NO 5 or SEQ ID NO 6 shows a considerable degree of homology to that of known members of the family of HDACs. For convenience, the polypeptide consisting of the amino acid sequence as set forth in SEQ ID NO:1 SEQ ID NO 5 or SEQ ID NO 6 will be designated as histone deacetylase 9 or HDAC9. Such a polypeptide, or a fragment thereof, is expressed in various normal tissues, for example, HDAC9 was present in normal testes, stomach, spleen, small intestine, placenta, liver, kidney, colon, lung, heart, and brain, as an approximately 3 kb transcript. HDAC9 was not detected in muscle, but this lane also did not hybridize GAPDH (FIG. 7). Fragments of the isolated polypeptide having an amino acid sequence as set forth in SEQ ID NO:1,SEQ ID NO 5 or SEQ ID NO 6 will comprise polypeptides comprising from about 5 to 148 amino acids, preferably from about 10 to about 143 amino acids, more preferably from about 20 to about 100 amino acids, and most preferably from about 20 to about 50 amino acids. Such fragments also form a part of the present invention. Preferably, fragments will encompass the catalytic domain, which is predicted to exist between amino acid number 1 to 390. In accordance with this aspect of the invention there are provided novel polypeptides of human origin as well as biologically, diagnostically or therapeutically useful fragments, variants and derivatives thereof, variants and derivatives of the fragments, and analogs of the foregoing.

In a second aspect, the invention provides an isolated DNA comprising a nucleotide sequence that encodes a polypeptide as mentioned above. In particular, the invention provides (1) an isolated DNA comprising the nucleotide sequence as set forth in SEQ ID NO:2; SEQ ID NO 7 or SEQ ID NO 8 (2) an isolated DNA comprising the nucleotide sequence set forth in SEQ ID NO:3; (3) an isolated DNA capable of hybridizing under high stringency conditions to the nucleotide sequence set forth in SEQ ID NO:3; and (4) an isolated DNA comprising the nucleotide sequence set forth in SEQ ID NO:4. Also provided are nucleic acid sequences comprising at least about 15 bases, preferably at least about 20 bases, more preferably a nucleic acid sequence comprising about 30 contiguous bases of SEQ ID NO:2, SEQ ID NO 7 or SEQ ID NO 8or SEQ ID NO:3. Also within the scope of the present invention are nucleic acids that are substantially similar to the nucleic acid with the nucleotide sequence as set forth in SEQ ID NO:2, SEQ ID NO 7 or SEQ ID NO 8 or SEQ ID NO:3. In a preferred embodiment, the isolated DNA takes the form of a vector molecule comprising at least a fragment of a DNA of the present invention, in particular comprising the DNA consisting of a nucleotide sequence as set forth in SEQ ID NO:2, SEQ ID NO 7 or SEQ ID NO 8 or SEQ ID NO:3.

A third aspect of the present invention encompasses a method for the diagnosis of conditions associated with abnormal regulation of gene expression which includes, but is not limited to, conditions associated with abnormal cell proliferation, cancer, atherosclerosis, inflammatory bowel disease, or psoriasis in a human which comprises detecting abnormal transcription of messenger RNA transcribed from the natural endogenous human gene encoding the novel polypeptide consisting of the amino acid sequence set forth in SEQ ID NO:1,SEQ ID NO 5 or SEQ ID NO 6 in an appropriate tissue or cell from a human, wherein such abnormal transcription is diagnostic of the human's affliction with such a condition. In particular, the said natural endogenous human gene encoding the novel polypeptide consisting of the amino acid sequence set forth in SEQ ID NO:1,SEQ ID NO 5 or SEQ ID NO 6 comprises the genomic nucleotide sequence set forth in SEQ ID NO:4. In one embodiment of the present invention, the diagnostic method comprises contacting a sample of said appropriate tissue or cell or contacting an isolated RNA or DNA molecule derived from that tissue or cell with an isolated nucleotide sequence of at least about 15-20 nucleotides in length that hybridizes under high stringency conditions with the isolated nucleotide sequence encoding the novel polypeptide having an amino acid sequence set forth in SEQ ID NOs:1., 5 or 6

Another embodiment of the assay aspect of the invention provides a method for the diagnosis of a condition associated with abnormal HDAC9 activity in a human, which comprises measuring the level of deacetylase activity in a certain tissue or cell from a human suffering from such a condition, wherein the presence of an abnormal level of deacetylase activity, relative to the level thereof in the respective tissue or cell of a human not suffering from a condition associated with abnormal HDAC activity, is diagnostic of the human's suffering from said condition.

In accordance with one embodiment of this aspect of the invention there are provided anti-sense polynucleotides that can regulate transcription of the gene encoding the novel HDAC9; in another embodiment, double stranded RNA is provided that can regulate the transcription of the gene encoding the novel HDAC9.

Another aspect of the invention provides a process for producing the aforementioned polypeptides, polypeptide fragments, variants and derivatives, fragments of the variants and derivatives, and analogs of the foregoing. In a preferred embodiment of this aspect of the invention there are provided methods for producing the aforementioned HDAC9 comprising culturing host cells having incorporated therein an expression vector containing an exogenously-derived nucleotide sequence encoding such a polynucleotide under conditions sufficient for expression of the polypeptide in the host cell, thereby causing expression of the polypeptide, and optionally recovering the expressed polypeptide. In a preferred embodiment of this aspect of the present invention, there is provided a method for producing polypeptides comprising or consisting of an amino acid sequence as set forth in SEQ ID NOs:1, 5 or 6 which comprises culturing a host cell having incorporated therein an expression vector containing an exogenously-derived polynucleotide encoding a polypeptide comprising or consisting of an amino acid sequence as set forth in SEQ ID NOs:1, 5 or 6 under conditions sufficient for expression of such a polypeptide in the host cell, thereby causing the production of an expressed polypeptide, and optionally recovering the expressed polypeptide. Preferably, in any of such methods the exogenously derived polynucleotide comprises or consists of the nucleotide sequence set forth in SEQ ID NOs:2, 7 or 8 the nucleotide sequence set forth in SEQ ID NO:3, or the nucleotide sequence set forth in SEQ ID NO:4. In accordance with another aspect of the invention there are provided products, compositions, processes and methods that utilize the aforementioned polypeptides and polynucleotides for, inter alia, research, biological, clinical and therapeutic purposes.

In certain additional preferred embodiments of this aspect of the invention there is provided an antibody or a fragment thereof which specifically binds to a polypeptide that comprises the amino acid sequence set forth in SEQ ID NOs:1, 5 or 6 i.e., all HDAC9 variants. In certain particularly preferred embodiments in this regard, the antibodies are highly selective for human HDAC9 polypeptides or portions of human HDAC9 polypeptides.

In a further aspect, an antibody or fragment thereof is provided that binds to a fragment or portion of the amino acid sequence set forth in SEQ ID NOs:1, 5 or 6.

In another aspect, methods of treating a condition in a subject, wherein the condition is associated with abnormal HDAC9 gene expression, an increase or decrease in the presence of HDAC9 polypeptide in a subject, or an increase or decrease in the activity of HDAC9 polypeptide, by the administration of an effective amount of an antibody that binds to a polypeptide with the amino acid sequence set out in SEQ ID NOs:1, 5 or 6., or a fragment or portion thereof to the subject are provided. Also provided are methods for the diagnosis of a disease or condition associated with abnormal HDAC9 gene expression or an increase or decrease in the presence of the HDAC9 in a subject, or an increase or decrease in the activity of HDAC9 polypeptide, which comprises utilizing conventional methodologies, including, for example, the H4 histone assay that was previously described (Inokoshi, J., Katagiri, M., Arima, S., Tanaka, H., Hayashi, M., Kim, Y. -B., Furumai, I., Yoshida, M., Horinouchi, S., Omura, S. (1999) Biochem. Biophys. Res. Com. 256, 372-376.).

In yet another aspect, the invention provides host cells which can be propagated in vitro, preferably vertebrate cells, in particular mammalian cells, or bacterial cells, which are capable upon growth in culture of producing a polypeptide that comprises the amino acid sequence set forth in SEQ ID NOs:1, 5 or 6 or fragments thereof, where the cells contain transcriptional control DNA sequences, where the transcriptional control sequences control transcription of RNA encoding a polypeptide with the amino acid sequence according to SEQ ID NOs:1, 5 or 6. or fragments thereof. This includes, but is not limited to, the propagation of HDAC9 in a plasmid and the production of DNA, RNA or protein in human or insect cells or bacteria using the endogenous HDAC9 promoter or any other transcriptional control sequence.

In yet another aspect of the present invention there are provided assay methods and kits comprising the components necessary to detect above-normal expression of polynucleotides encoding a polypeptide comprising an amino acid sequence as set forth in SEQ ID NOs:1, 5 or 6., or polypeptides comprising an amino acid sequence set forth in SEQ ID NOs:1, 5 or 6., or fragments thereof, in body tissue samples derived from a patient, such kits comprising e.g., antibodies that bind to a polypeptide comprising an amino acid sequence set forth in SEQ ID NOs:1, 5 or 6 or to fragments thereof, or oligonucleotide probes that hybridize with polynucleotides of the invention. In a preferred embodiment, such kits also comprise instructions detailing the procedures by which the kit components are to be used.

In another aspect, the invention is directed to use of a polypeptide comprising an amino acid sequence set forth in SEQ ID NOs:1, 5 or 6. or fragment thereof, polynucleotide encoding such a polypeptide or a fragment thereof, or antibody that binds to said polypeptide comprising an amino acid sequence set forth in SEQ ID NOs:1, 5 or 6. or a fragment thereof in the manufacture of a medicament to treat diseases associated with abnormal HDAC activity or gene expression.

Another aspect is directed to pharmaceutical compositions comprising a polypeptide comprising or consisting of an amino acid sequence set forth in SEQ ID NOs:1, 5 or 6. or fragment thereof, a polynucleotide encoding such a polypeptide or a fragment thereof, or antibody that binds to such a polypeptide or a fragment thereof, in conjunction with a suitable pharmaceutical carrier, excipient or diluent, for the treatment of diseases associated with abnormal HDAC activity or gene expression.

In another aspect, the invention is directed to methods for the identification of molecules that can bind to a polypeptide comprising an amino acid sequence set forth in SEQ ID NOs:1, 5 or 6. and/or modulate the activity of a polypeptide comprising an amino acid sequence set forth in SEQ ID NOs:1, 5 or 6. or molecules that can bind to nucleic acid sequences that modulate the transcription or translation of a polynucleotide encoding a polypeptide comprising an amino acid sequence set forth in SEQ ID NOs:1, 5 or 6. Such methods are disclosed in, e.g., U.S. Pat. Nos. 5,541,070; 5,567,317; 5,593,853; 5,670,326; 5,679,582; 5,856,083; 5,858,657; 5,866,341; 5,876,946; 5,989,814; 6,010,861; 6,020,141; 6,030,779; and 6,043,024, all of which are incorporated by reference herein in their entirety. Molecules identified by such methods also fall within the scope of the present invention.

In a related aspect, the invention is directed to use of the novel HDAC9 to identify associated proteins in HDAC biologically relevant complexes. At present, the proteins that associate with HDAC9 are not known. However, these may be characterized by determining whether HDAC9 associates with proteins that have been previously shown to interact with other HDACs (see Introduction). For example, components of HDAC9 complexes may be determined using conventional methods, including co-immunoprecipitation (see Example 9).

In yet another aspect, the invention is directed to methods for the introduction of nucleic acids of the invention into one or more tissues of a subject in need of treatment with the result that one or more proteins encoded by the nucleic acids are expressed and or secreted by cells within the tissue.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

FIG. 1 shows the 1156 bp open reading frame that was identified using GENFAM (proprietary software) and used to search databases for the complete HDAC9 cDNA sequence. The respective ORF (SEQ ID NO:3) starts at nucleotide position no. 1 and ends at nucleotide position no. 1156.

FIGS. 2A and 2B show the full length cDNA sequence (SEQ ID NO:2) of HDAC9 and the amino acid sequence (SEQ ID NO:1), respectively. The full length cDNA sequence starts at nucleotide position no. 1 and ends at nucleotide position 2022.

FIG. 3 shows the genomic DNA sequence in silico (AL022328) (SEQ ID NO:4), aligned with the sequence of clone 198929/HDAC9. The alignment was produced using proprietary software (Novartis Pharmaceuticals, Summit, N.J.).

FIG. 4 is a depiction of the alignment of HDAC9 predicted peptide and S. pombe Hda1 peptide. The query is HDAC9 peptide and the subject is S. pombe Hda1 peptide. The alignment was produced using Clustalw algorhithm (Higgins, D. G., Thompson, J. D., Gibson, T. J. (1996) Using CLUSTAL for multiple sequence alignments. Methods Enzymol 266, 383-402).

FIG. 5 shows the alignment of HDAC1 and HDAC9v1 and locations of the putative catalytic domain amino acids and Rb-binding domain. Catalytic domain amino acids are boxed and putative Rb domain amino acids are contained within crosshatched boxes. The alignment was produced using Clustalw algorhithm (Higgins, D. G., Thompson, J. D., Gibson, T. J. (1996) Using CLUSTAL for multiple sequence alignments. Methods Enzymol 266, 383-402).

FIG. 6 shows the alignment of HDACs 1-9v1. The alignment was produced using Clustalw algorhithm (Higgins, D. G., Thompson, J. D., Gibson, T. J. (1996) Using CLUSTAL for multiple sequence alignments. Methods Enzymol 266, 383-402).

FIG. 7 shows the Northern analysis of HDAC9. (A) Northern blot analysis of the distribution of HDAC9 in normal human tissues. GAPDH was hybridized to the same blot as a control for RNA loading. (B) Northern blot analysis of HDAC9 in matched tumor and normal tissues. GAPDH was hybridized to the same blot as a control for RNA loading.

FIG. 8 shows Real Time PCR analysis of the distribution of HDAC9 in normal human tissues and cell lines relative to 18S ribosomal RNA. RNA from the human lung carcinoma cell line, A549 was used as an internal control.

FIG. 9 shows the alignment of HDAC9v1 with class II HDACs (HDACs 4, 5, 6, 7). The alignment was produced using Clustalw algorhithm (Higgins, D. G., Thompson, J. D., Gibson, T. J. (1996) Using CLUSTAL for multiple sequence alignments. Methods Enzymol 266,383-402). Catalytic domain amino acids are boxed.

FIG. 10 shows the alignment of HDAC9v1 with class I HDACs (HDACs 1, 2, 3, 8). The alignment was produced using Clustalw algorhithm (Higgins, D. G., Thompson, J. D., Gibson, T. J. (1996) Using CLUSTAL for multiple sequence alignments. Methods Enzymol 266, 383-402). Catalytic domain amino acids are boxed.

FIG. 11 There are threee HDAC9 sequence variants (HDAC9v1, HDAc9v2, and HDAC9v3). HDAC9v1 and HDA9v2 were found by searching the human EST database and HDAC9v3 was found as a predicted transcript in the Celera Sequence database. (A) shows an alignment of the 3 HDAC9 variant peptide sequences. (B) shows a schematic of class I and class II HDAC peptide sequences. Catalytic domains are in filled boxes and putative LXCXE motifs are in open boxes (C) is a schematic of the genomic structures of HDAC9v1 and HDAC9v2. Exons are shown as filled boxes and introns are shown as lines between the filled boxes. Lengths of boxes and lines represent the lengths of exons and introns.

FIG. 12 shows that HDAC9 is an enzymatically active histone deacetylase. (A) HDAC9 catalytic activity is comparable to the activity of HDAC3 and HDAC4. (B) shows that HDAC1 was more efficient than HDAC3, HDAC4, and HDAC9 at deacetylating the histone substrate in this assay.

FIG. 13 shows that HDAC9 is a nuclear protein and shows that HDAC9-flag is in vitro translated.

FIG. 14 shows DNA and peptide sequences for HDAC9v3 and HDAC9v2.

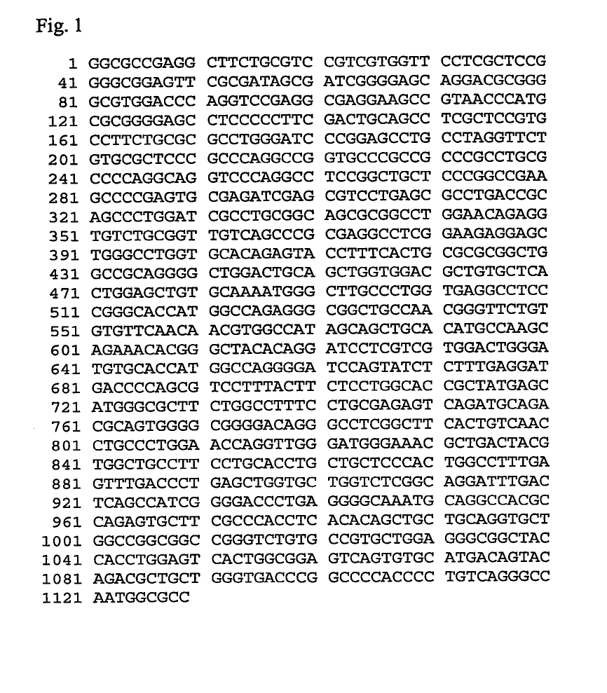

### DETAILED DESCRIPTION OF THE INVENTION

All patent applications, patents and literature references cited herein are hereby incorporated by reference in their entirety.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The following abbreviations used throughout the disclosure are listed herein below: histone deacetylase (HDAC), histone deacetylase-like protein (HDLP)

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The percentage of identity between the substantially similar nucleotide sequence and the reference nucleotide sequence desirably is at least 80%, more desirably at least 85%, preferably at least 90%, more preferably at least 95%, still more preferably at least 99%. Sequence comparisons are carried out using Clustalw (see, for example, Higgins, D. G. et al. Methods Enzymol. 266:383-402 (1996)). Clustalw alignments were performed using default parameters.

A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C., yet still encodes a functionally equivalent gene product.

“Elevated transcription of mRNA” refers to a greater amount of messenger RNA transcribed from the natural endogenous human gene encoding the novel polypeptide of the present invention present in an appropriate tissue or cell of an individual suffering from a condition associated with abnormal HDAC9 activity than in a subject not suffering from such a disease or condition; in particular at least about twice, preferably at least about five times, more preferably at least about ten times, most preferably at least about 100 times the amount of mRNA found in corresponding tissues in humans who do not suffer from such a condition. Such elevated level of mRNA may eventually lead to increased levels of protein translated from such mRNA in an individual suffering from a condition associated with abnormal cellular proliferation as compared with a healthy individual. It is also understood that “elevated transcription of mRNA” may refer to a greater amount of messenger RNA transcribed from genes the expression of which is modulated by HDAC9 either alone or in combination with other molecules.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and the like.

“Heterologous” as used herein means “of different natural origin” or represent a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g. a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.”

“Plasmids” generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” refers to DNA sequences, such as initiator sequences, enhancer sequences, and promoter sequences, which induce, repress, or otherwise control the transcription of protein encoding nucleic acid sequences to which they are operably linked.

As used herein, “human transcriptional control sequences” are any of those transcriptional control sequences normally found associated with the human gene encoding the novel HDAC9 polypeptide of the present invention as it is found in the respective human chromosome. It is understood that the term may also refer to transcriptional control sequences normally found associated with human genes the expression of which is modulated by HDAC9 either alone or in combination with other molecules.

As used herein, “non-human transcriptional control sequence” is any transcriptional control sequence not found in the human genome.

The term “polypeptide” is used interchangeably herein with the terms “polypeptides” and “protein(s)”.

As used herein, a “chemical derivative” of a polypeptide of the invention is a polypeptide of the invention that contains additional chemical moieties not normally a part of the molecule. Such moieties may improve the molecule's solubility, absorption, biological half life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16th ed., Mack Publishing Co., Easton, Pa. (1980).

As used herein, “HDAC9” refers to the amino acid sequences of substantially purified HDAC9 obtained from any species, particularly mammalian, including bovine, ovine, porcine, murine, equine, and preferably human, from any source, whether natural, synthetic, semi-synthetic, or recombinant.

As used herein, “HDAC activity”, including “HDAC9 activity” refers to the ability of an HDAC polypeptide to deacetylate histone proteins, including ³H-labeled H4 histone peptide. Such activity may be measured according to conventional methods, for example as described in Inokoshi, J., Katagiri, M., Arima, S., Tanaka, H., Hayashi, M., Kim, Y.- B., Furunai, R., Yoshida, M., Horinouchi, S., and Omura, S. (1999) Biochem. Biophys. Res. Com. 256, 372-376. A biologically “active” protein refers to a protein having structural, regulatory, or biochemical functions of a naturally occurring molecule.

The term “agonist”, as used herein, refers to a molecule which when bound to HDAC9, causes a change in HDAC9 which modulates the activity of HDAC9. Agonists may include proteins, nucleic acids, carbohydrates, or any other molecules that bind to HDAC9.

The terms “antagonist” or “inhibitor” as used herein, refer to a molecule which when bound to HDAC9, blocks or modulates the biological activity of HDAC9. Antagonists and inhibitors may include proteins, nucleic acids, carbohydrates, or any other molecules, natural or synthetic that bind to HDAC9.

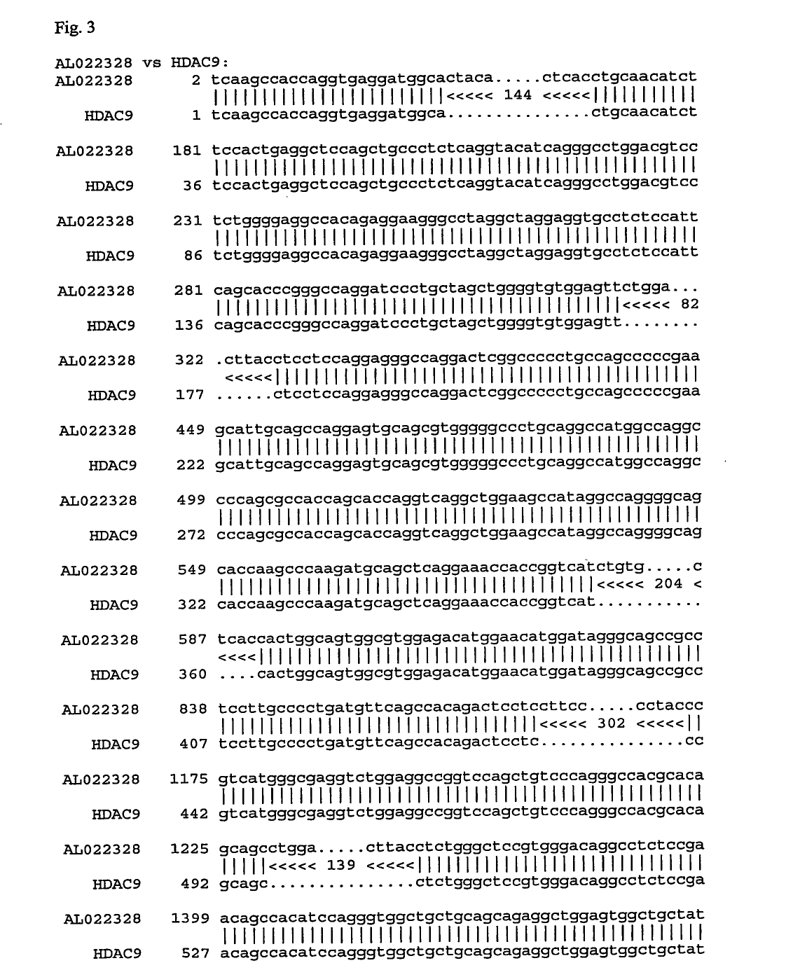

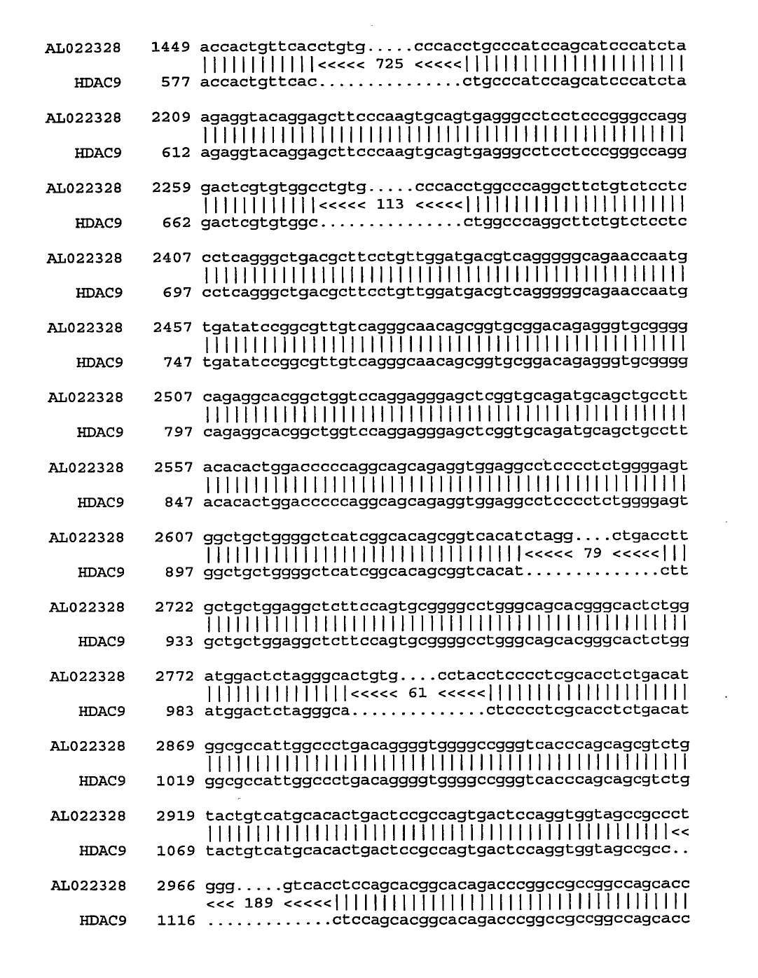

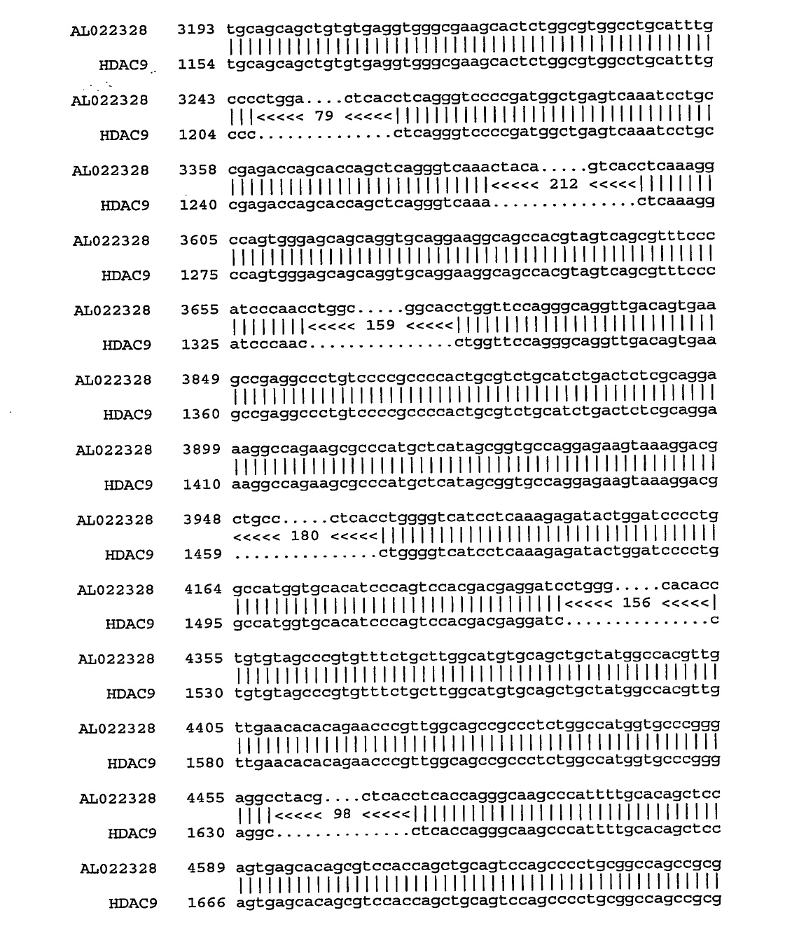

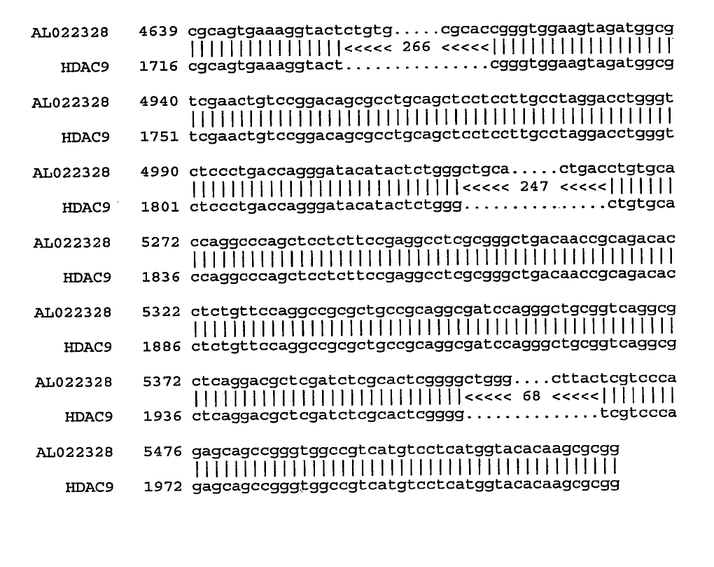

HDAC9 was identified using proprietary computer software called GENFAM to search for new human sequences that are related to histone deacetylases in the Celera Human Genome Database, Incyte LIESEQ® database and the public High Throughput Genomic database. An 1156 bp open reading frame (ORF) was identified and used to search a database of sequenced clones from pan-tissue and dorsal root ganglion cDNA libraries. Four clones were found to contain the ORF (M6, K10, P3, F23), two from each library. Of these clones, M6, from the pan-tissue library was determined to be the most complete cDNA as a result of sequence analysis and in vitro translation. BLAST (Altshul S. F. et al Nucleic Acid Res 25:3389-402 (1997)) was used to search the Genbank database using cDNA clone M6. Genomic sequence AL022328 was found to contain exons that were identical in sequence to the M6 cDNA. A Clustalw alignment of the antisense sequence of HDAC9 (2022 to 8) with genomic sequence AL022328 is shown in FIG. 3. The first 7 bases of the HDAC9 predicted cDNA are not aligned, presumably because they occur following the next intron and this sequence was probably too short for the software to determine an alignment. The sequence of cDNA clone M6 was confrined by automated DNA sequencing (ACGT, Inc., Northbrook, Ill.). Based upon the predicted cDNA sequence from genomic sequence AL022328, 44 bases were missing from the N-terminus of M6. This sequence was subsequently added by PCR.

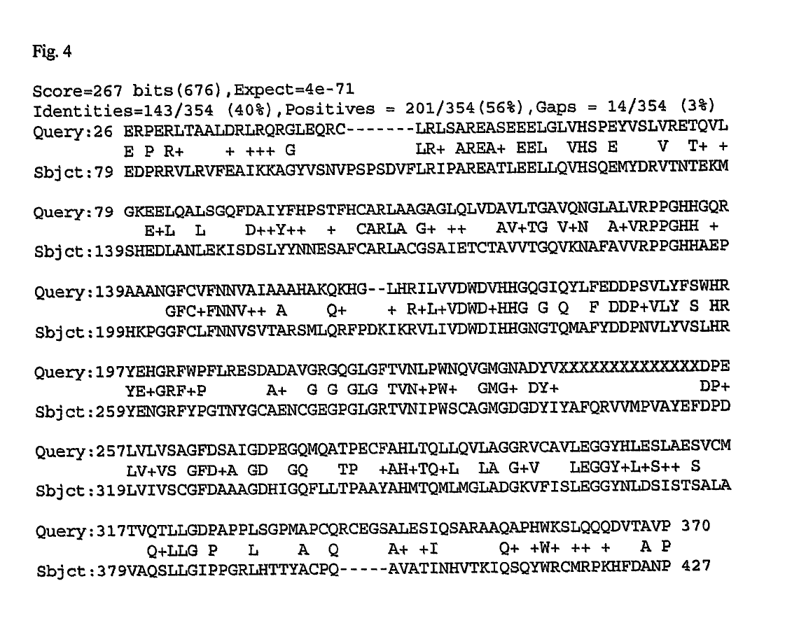

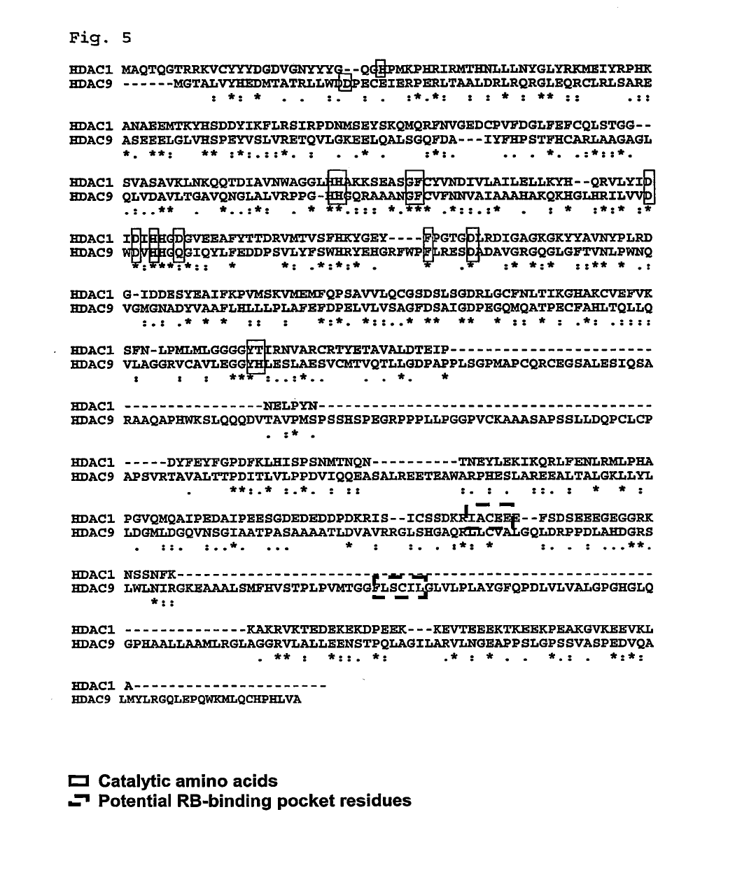

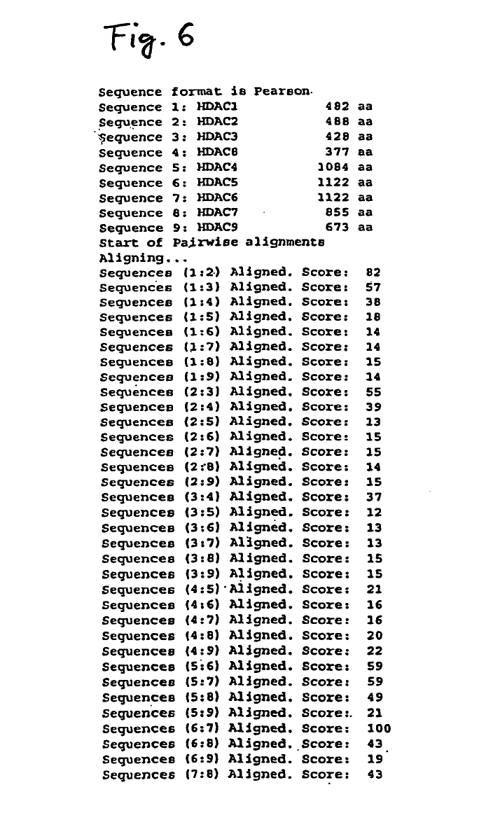

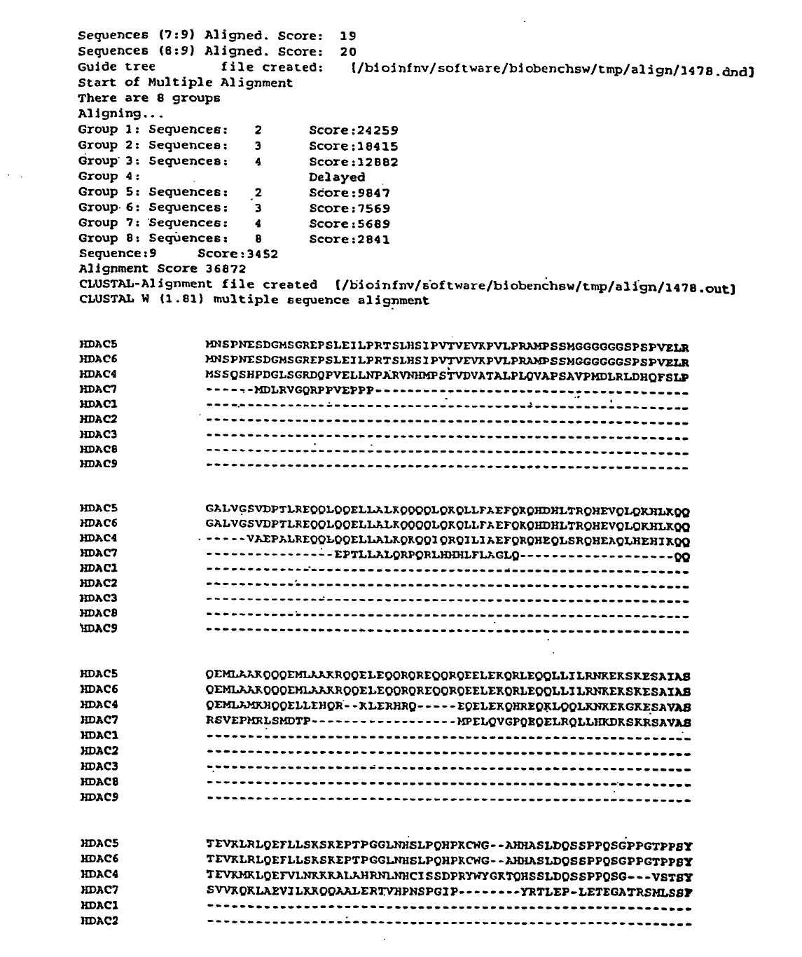

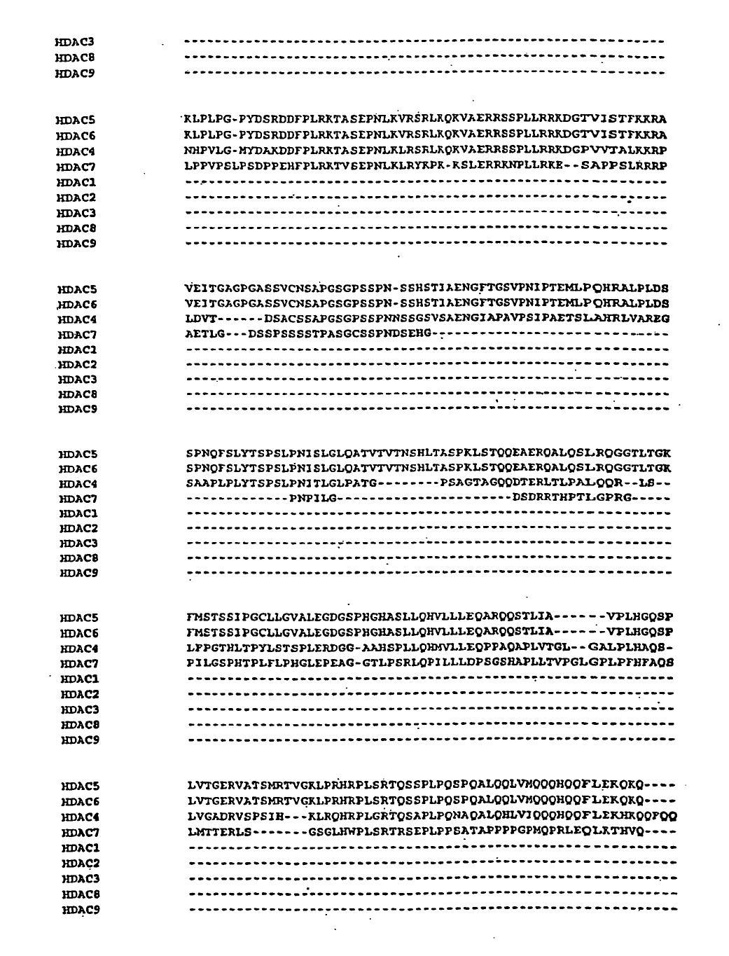

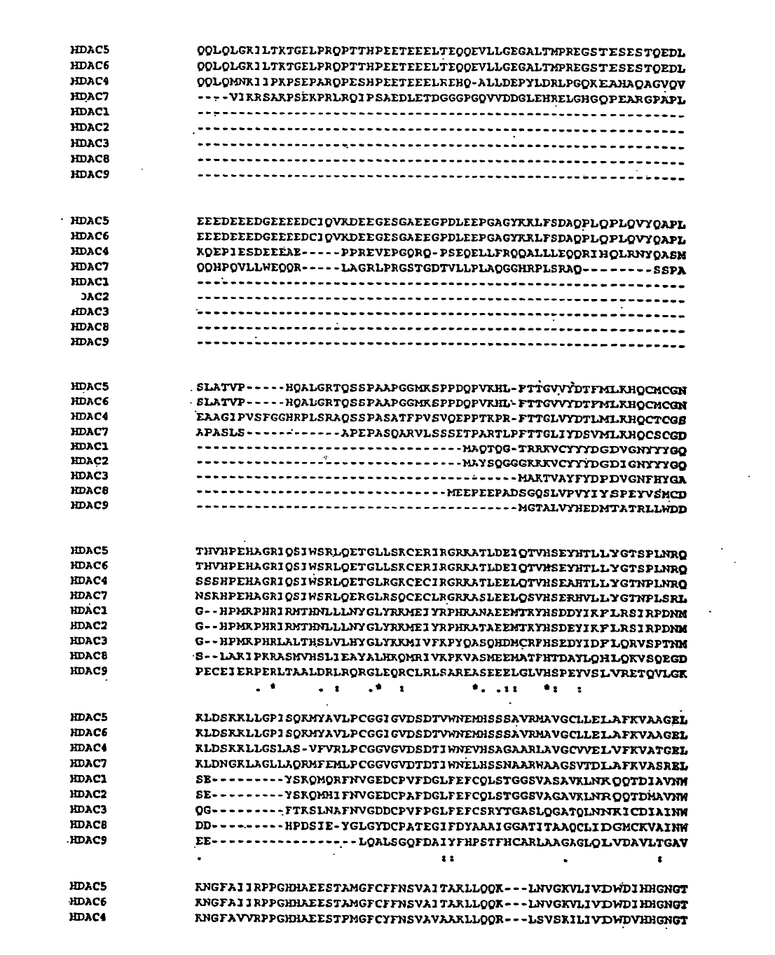

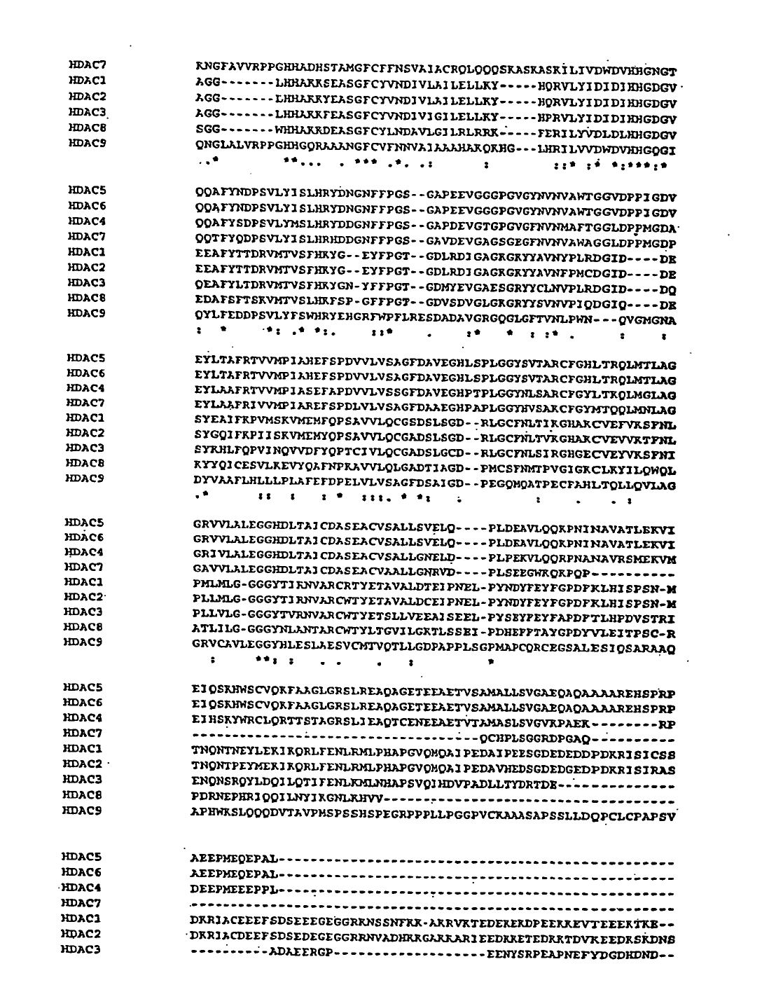

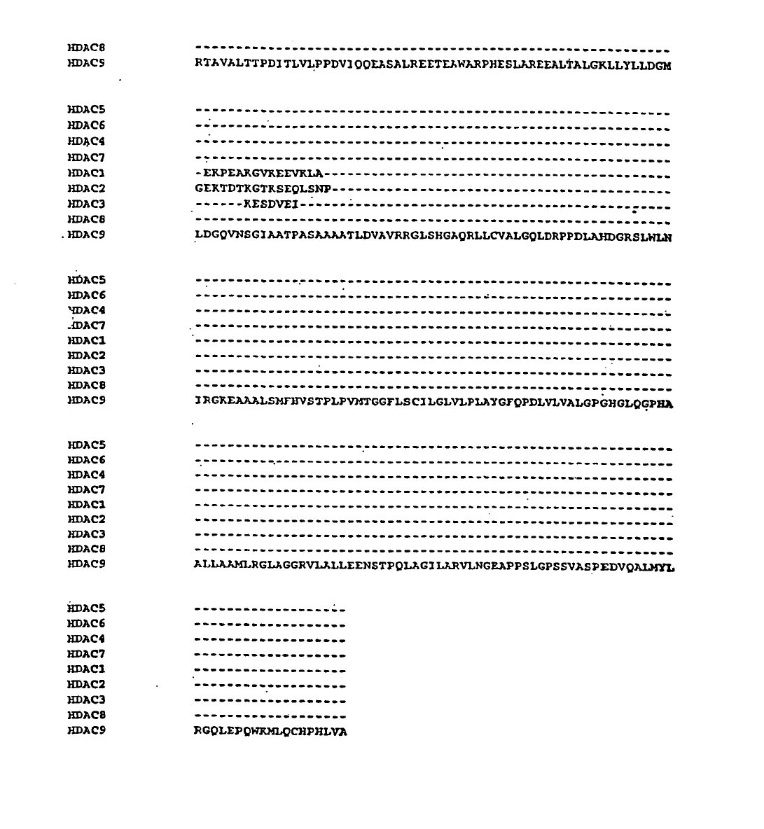

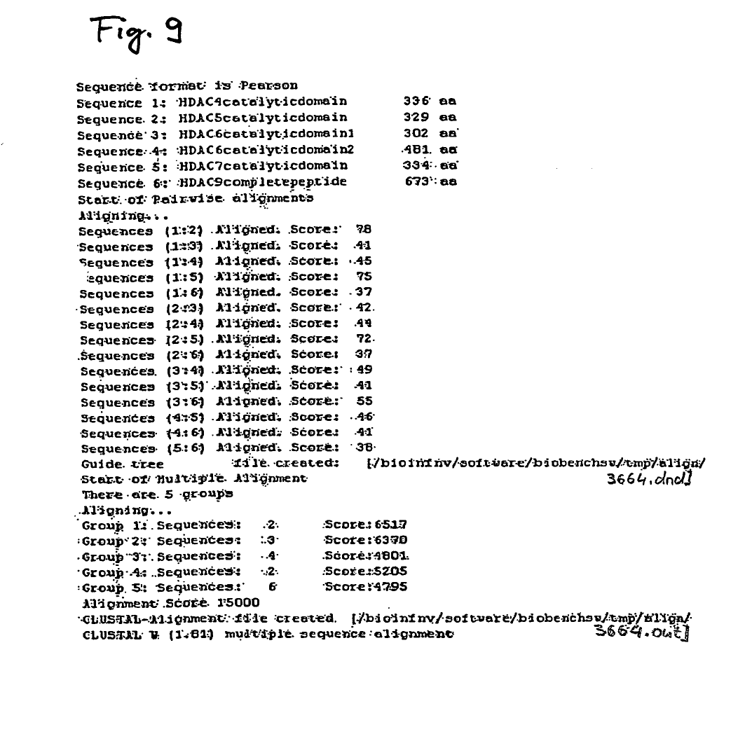

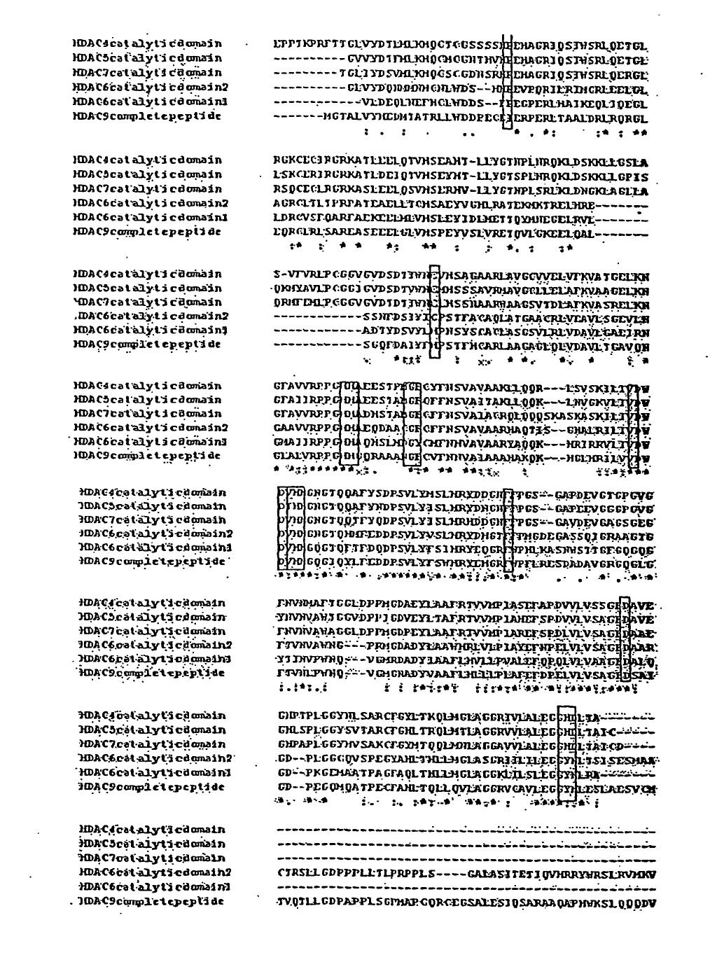

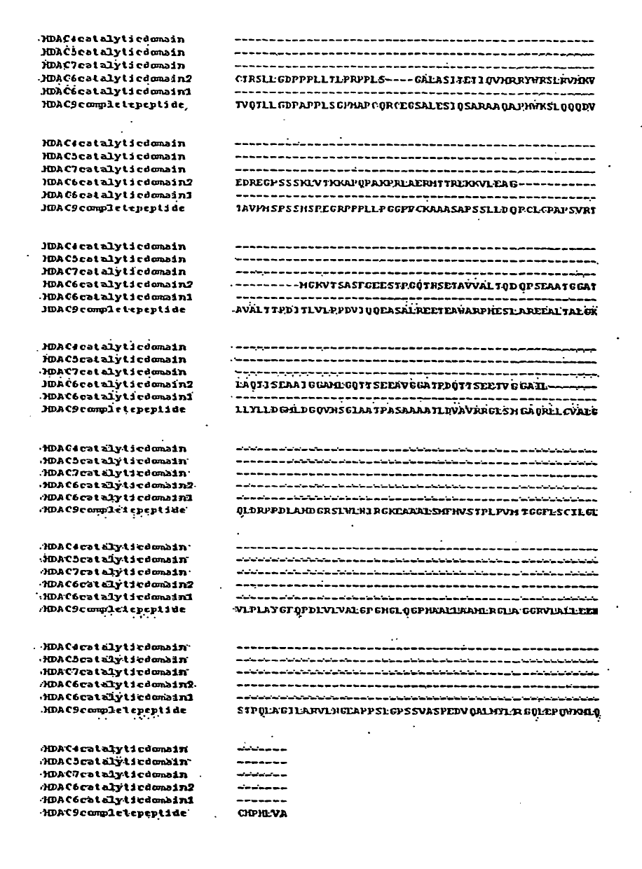

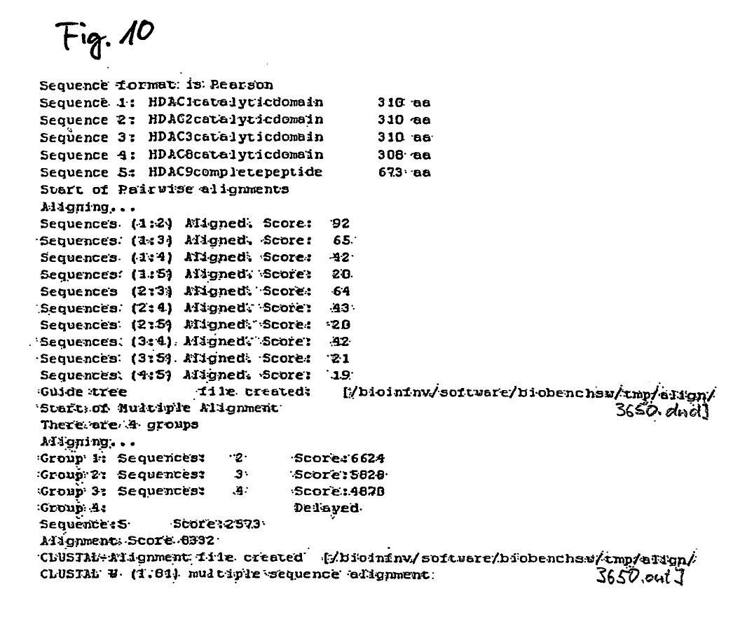

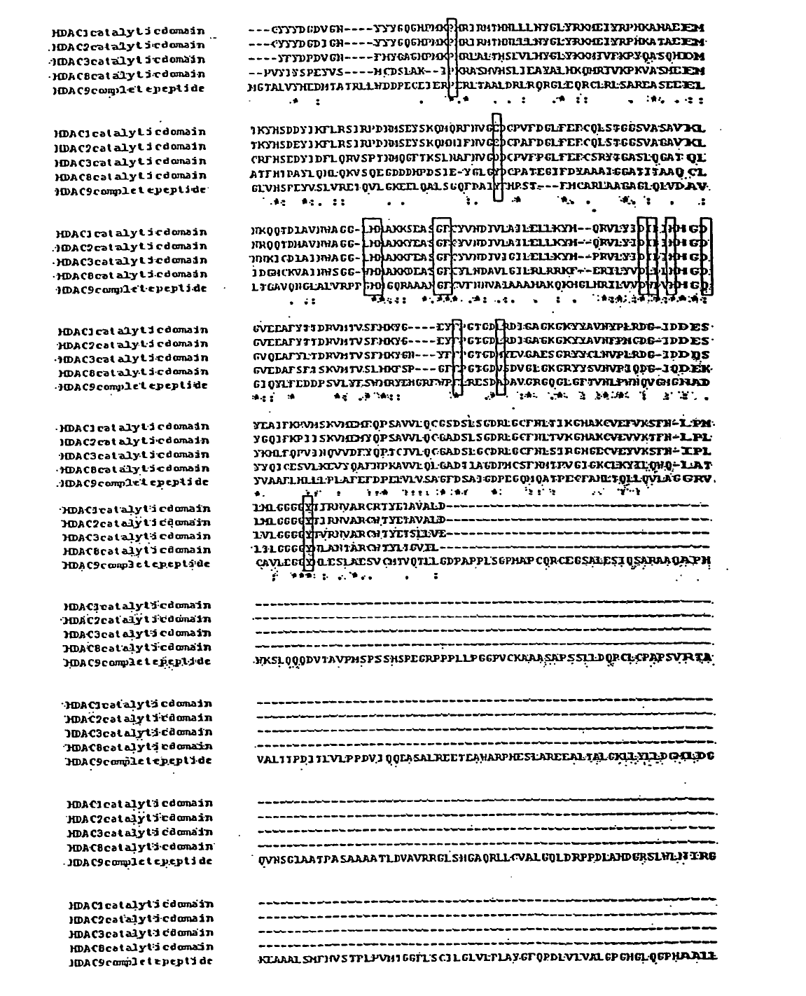

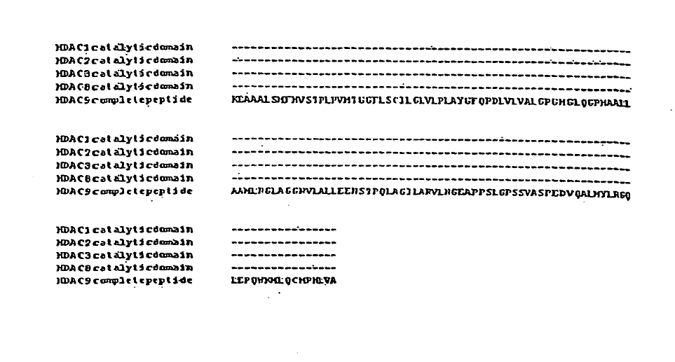

The full length cDNA for HDAC9 predicts a protein of 673 amino acids. The HDAC9 cDNA sequence is 2022 base pairs in length. In order to determine the percent similarity of HDAC9 to other known HDACs, a Clustalw multiple sequence alignment was performed using complete peptide sequences for HDACs 1-9. HDAC9 is most similar in peptide sequence to human HDAC6 at 37%. The Clustalw alignment of HDAC9 with class II HDACs is shown in FIG. 9. HDAC9 was also 40% similar to a yeast class II sequence hda1 from S. pombe. The Clustalw alignment of human HDAC9 and S. pombe is shown in FIG. 4. HDAC9 was less similar to class I HDACs (≦18%). The Clustalw alignment of HDAC9 to class I HDACs is shown in FIG. 10. HDAC9 possesses a putative catalytic domain which encompasses approximately 317 aa (˜6 to 323) based upon alignments of HDAC9 with the putative catalytic domains of all of the other known HDACs. To identify the catalytic domain of HDAC9, Clustalw alignments were performed separately using HDAC9 complete peptide and catalytic domain sequences from class I HDACs (1-3 and 8) or class II HDACs (4-7). 13 amino acids were previously shown to confer deacetylase activity, based upon inactivation by single amino acid mutations and the three dimensional structure formed by a complex of HDAC-like protein (HDLP), Zn2+ and HDAC inhibitors (Finnin, M. S., Doniglan, J. R., Cohen, A., Richon, V. M., Rifkind, R. a., Marks, P. A., Breslow, R., and Pavletich, N. P. (1999) Structures of a histone deacetylase homologue bound to TSA and SAHA inhibitors. Nature 401, 188-193). These 13 amino acids include Pro 22, His 131, His 132, Gly 140, Phe 141, Asp 166, Asp 168, His 170, Asp 173, Phe 198, Asp 258, Leu 265, and Tyr 297. 12 out of 13 of these amino acids are conserved in HDAC9. The amino acid that is not conserved is Leu 265. This hydrophobic residue forms part of the TS binding pocket and is replaced in HDAC9 with Glu at amino acid 272. Leu 265 is replaced with Met in HDAC8 and Lys in HDAC 6 domain 1. This suggests that this residue is not highly conserved and need not be identical to other HDACs. The second residue that differs from HDLP, HDAC1, and HDAC2, Asp 173 is substituted with Gln at position 177 in HDAC9, a difference that is also present in the HDAC6 catalytic domain 1. Furthermore, Asp 173 is substituted with Asn in HDACs 4, 5, 6 (domain 2), and 7. This evidence suggests that these Asp173 substitutions do not affect HDAC activity.

An amino acid sequence motif was previously found to be important for the binding of HDACs 1 and 2 to retinoblastoma protein (Rb). Complexes of HDACs 1 and 2 and Rb induce repression of E2F responsive promoters (Brehm, A., Miska, E. A., McCance, D. J., Reid, J. L., Bannister, A. J., and Kouzarides, T. (1998) Nature 391, 597-601). An Rb-binding motif fits the sequence model LXCXE, where “X” can be any amino acid. The LXCXE domain has been found to be dispensible for growth suppression function of Rb, but is necessary for HDAC binding (Chen, T. -T. and Wang, J. Y. J. (2000) Mol. Cell Biol. 20, 5571-5580). The Rb-binding domain that was previously determined in HDAC1 is located from amino acid 414 to amino acid 419 and is the sequence IACEE. So far, it has not been determined whether other HDACs are capable of binding to Rb. However, HDAC 9 contains a putative Rb-binding motif, LSCIL, that aligned with HDAC1 IACEE and is located between amino acids 560 and 564. Co-immunoprecipitation of HDAC9 with Rb is one strategy that may be used to validate the function of this motif in HDAC9.

As a member of the HDAC family, HDAC9 could form biologically relevant complexes with proteins and display functions that have been described for other HDACs. For example, it is likely to be involved in the regulation of transcription as a component of complexes that are involved in transcriptional repression that is mediated through interactions of HDACs with multi-protein complexes and which requires deacetylase activity. Thus, increased activity or expression of HDAC9 may be associated with numerous pathological conditions, including but not limited to, abnormal cell proliferation, cancer, atherosclerosis, inflammatory bowel disease, host inflammatory or immune response, or psoriasis.

Thus, the DNA/amino acid sequence and predicted structure of HDAC9 will be useful for designing agents (e.g. antagonists or inhibitors) useful to ameliorate conditions associated with abnormal HDAC activity. These may include, for example, antiproliferative or antiinflammatory agents either through the use of small molecules or proteins (e.g. antibodies) directed against it or associated proteins in HDAC transcription repressor complexes. In addition, protein derived from the HDAC9 sequence may also be used as a therapeutic to modify host cell proliferative or inflammatory responses.

To determine the expression pattern of the novel polypeptide, a panel of mRNAs from a variety of human tissues is subjected to Northern analysis. Data indicate that HDAC9 is expressed in human tissues, being detectable in brain, colon, heart, kidney, liver, placenta, small intestine, spleen, stomach and testes. Thus, HDAC9 represents a transcribed gene.

Therefore, in one aspect, the present invention relates to a novel histone deacetylase (HDAC). As outlined above, HDAC9 is clearly a member of the HDAC family since it is highly similar to other HDAC proteins in the hda1 class II HDACs. It also shares many similarities with the HDAC family.

The present invention relates to an isolated polypeptide comprising the amino acid sequence set forth in SEQ ID NO:1. For example, such a polypeptide may be a fusion protein including the amino acid sequence of the novel HDAC9. In another aspect the present invention relates to an isolated polypeptide consisting of the amino acid sequence set forth in SEQ ID NO:1, which is, in particular, the novel HDAC9.

The invention includes nucleic acid or nucleotide molecules, preferably DNA molecules, in particular encoding the novel HDAC9. Preferably, an isolated nucleic acid molecule, preferably a DNA molecule, of the present invention encodes a polypeptide comprising the amino acid sequence set forth in SEQ ID NO:1 SEQ ID NO 5 or SEQ ID NO 6. Likewise preferred is an isolated nucleic acid molecule, preferably a DNA molecule, encoding a polypeptide consisting of the amino acid sequence set forth in SEQ ID NO:1, SEQ ID NO 5 or SEQ ID NO 6. Such a nucleic acid or nucleotide, in particular such a DNA molecule, preferably comprises a nucleotide sequence selected from the group consisting of (1) the nucleotide sequence as set forth in SEQ ID NO:2,, 7 or 8 which is the complete cDNA sequence encoding the polypeptide consisting of the amino acid sequence set forth in SEQ ID NO:1, 5 and 6, respectively, (2) the nucleotide sequence set forth in SEQ ID NO:3, which corresponds to the open reading frame of the cDNA sequence set forth in SEQ ID NO:2; (3) a nucleotide sequence capable of of hybridizing under high stringency conditions to a nucleotide sequence set forth in SEQ ID NO:3; and (4) the nucleotide sequence set forth in SEQ ID NO:4, which corresponds to the endogenous genomic human DNA encoding the polypeptide consisting of the amino acid sequence set forth in SEQ ID NO:1. Such hybridization conditions may be highly stringent or less highly stringent, as described above. In instances wherein the nucleic acid molecules are deoxyoligonucleotides (“oligos”), highly stringent conditions may refer, e.g., to washing in 6× SSC/0.05% sodium pyrophosphate at 37° C. (for 14-base oligos), 48° C. (for 17-base oligos), 55° C. (for 20-base oligos), and 60° C. (for 23-base oligos). Suitable ranges of such stringency conditions for nucleic acids of varying compositions are described in Krause and Aaronson (1991), Methods in Enzymology, 200:546-556 in addition to Maniatis et al., cited above.

These nucleic acid molecules may act as target gene antisense molecules, useful, for example, in target gene regulation and/or as antisense primers in amplification reactions of target gene nucleic acid sequences. Further, such sequences may be used as part of ribozyme and/or triple helix sequences, also useful for target gene regulation. Still further, such molecules may be used as components of diagnostic methods whereby the presence of an allele causing a disease associated with abnormal HDAC9 expression or activity, for example, abnormal cell proliferation, cancer, atherosclerosis, inflammatory bowel disease, host inflammatory or immune response, or psoriasis, may be detected.

The invention also encompasses (a) vectors that contain at least a fragment of any of the foregoing nucleotide sequences and/or their complements (i.e., antisense); (b) vector molecules, preferably vector molecules comprising transcriptional control sequences, in particular expression vectors, that contain any of the foregoing coding sequences operatively associated with a regulatory element that directs the expression of the coding sequences; and (c) genetically engineered host cells that contain a vector molecule as mentioned herein or at least a fragment of any of the foregoing nucleotide sequences operatively associated with a regulatory element that directs the expression of the coding sequences in the host cell. As used herein, regulatory elements include, but are not limited to, inducible and non-inducible promoters, enhancers, operators and other elements known to those skilled in the art that drive and regulate expression. Preferably, host cells can be vertebrate host cells, preferably mammalian host cells, such as human cells or rodent cells, such as CHO or BHK cells. Likewise preferred, host cells can be bacterial host cells, in particular E.coli cells.

Particularly preferred is a host cell, in particular of the above described type, which can be propagated in vitro and which is capable upon growth in culture of producing an HDAC9 polypeptide, in particular a polypeptide comprising or consisting of an amino acid sequence set forth in SEQ ID NO:1, wherein said cell contains some fragment or complete sequence of HDAC9 coding sequence in a construct that is controlled by one or more transcriptional control sequences that is not a transcriptional control sequence of the natural endogeneous human gene encoding said polypeptide, wherein said one or more transcriptional control sequences control transcription of a DNA encoding said polypeptide. Possible transcriptional control sequences include, but are not limited to, bacterial or viral promoter sequences.

The invention includes the complete sequence of the gene as well as fragments of any of the nucleic acid sequences disclosed herein. Fragments of the nucleic acid sequences encoding the novel HDAC9 polypeptide may be used as a hybridization probe for a cDNA library to isolate other genes which have a high sequence similarity to the HDAC9 gene or similar biological activity. Probes of this type preferably have at least about 30 bases and may contain, for example, from about 30 to about 50 bases, about 50 to about 100 bases, about 100 to about 200 bases, or more than 200 bases. The probe may also be used to identify a cDNA clone corresponding to a full length transcript and a genomic clone or clones that contain the complete HDAC9 gene including regulatory and promoter regions, exons, and introns. An example of a screen comprises isolating the coding region of the HDAC9 gene by using the known DNA sequence to synthesize an oligonucleotide probe. Labeled oligonucleotides having a sequence complementary to that of the gene of the present invention may be used to screen a library of human cDNA, genomic DNA or mRNA to determine which members of the library to which the probe hybridizes.

In addition to the gene sequences described above, homologs of such sequences, as may, for example, be present in other species, may be identified and may be readily isolated, without undue experimentation, by molecular biological techniques well known in the art. Further, there may exist genes at other genetic loci within the genome that encode proteins which have homology to one or more domains of such gene products. These genes may also be identified via similar techniques. For example, the isolated nucleotide sequence of the present invention encoding the novel HDAC9 polypeptide may be labeled and used to screen a cDNA library constructed from mRNA obtained from the organism of interest. Hybridization conditions will be of a lower stringency when the cDNA library is derived from an organism different from the type of organism from which the labeled sequence was derived. Alternatively, the labeled fragment may be used to screen a genomic library derived from the organism of interest, again, using appropriately stringent conditions. Such low stringency conditions will be well known to those of skill in the art, and will vary predictably depending on the specific organisms from which the library and the labeled sequences are derived. For guidance regarding such conditions see, for example, Sambrook et al. cited above.

Further, a previously unknown differentially expressed gene-type sequence may be isolated by performing PCR using two degenerate oligonucleotide primer pools designed on the basis of amino acid sequences within the gene of interest. The template for the reaction may be cDNA obtained by reverse transcription of mRNA prepared from human or non-human cell lines or tissue known or suspected to express a differentially expressed gene allele. The PCR product may be subcloned and sequenced to ensure that the amplified sequences represent the sequences of a differentially expressed gene-like nucleic acid sequence. The PCR fragment may then be used to isolate a complete cDNA clone by a variety of conventional methods. For example, the amplified fragment may be labeled and used to screen a bacteriophage cDNA library. Alternatively, the labeled fragment may be used to screen a genomic library.

PCR technology may also be utilized to isolate full length cDNA sequences. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, supra.

In cases where the gene identified is the normal, or wild type, gene, this gene may be used to isolate mutant alleles of the gene. Such an isolation is preferable in processes and disorders which are known or suspected to have a genetic basis. Mutant alleles may be isolated from individuals either known or suspected to have a genotype which contributes to disease symptoms related to abnormal HDAC activity, including, but not limited to, conditions such as abnormal cell proliferation, cancer, atherosclerosis, inflammatory bowel disease, host inflammatory or immune response, or psoriasis. Mutant alleles and mutant allele products may then be utilized in the diagnostic assay systems described below.

A cDNA of the mutant gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant gene to that of the normal gene, the mutation(s) responsible for the loss or alteration of function of the mutant gene product can be ascertained.

Alternatively, a genomic or CDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. (For screening techniques, see, for example, Harlow, E. and Lane, eds., 1988, “Antibodies: A Laboratory Manual”, Cold Spring Harbor Press, Cold Spring Harbor.) In cases where the mutation results in an expressed gene product with altered function (e.g., as a result of a missense mutation), a polyclonal set of antibodies are likely to cross-react with the mutant gene product. Library clones detected via their reaction with such labeled antibodies can be purified and subjected to sequence analysis as described above.

The present invention includes those proteins encoded by nucleotide sequences set forth in any of SEQ ID NOs:2, 3, 4, 7 or 8 in particular, a polypeptide that is or includes the amino acid sequence set out in SEQ ID NO:1, 5 or 6 or fragments thereof.

Furthermore, the present invention includes proteins that represent functionally equivalent gene products. Such an equivalent differentially expressed gene product may contain deletions, additions or substitutions of amino acid residues within the amino acid sequence encoded by the differentially expressed gene sequences described, above, but which result in a silent change, thus producing a functionally equivalent differentially expressed gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent,” as utilized herein, may refer to a protein or polypeptide capable of exhibiting a substantially similar in vivo or in vitro activity as the endogenous differentially expressed gene products encoded by the differentially expressed gene sequences described above. “Functionally equivalent” may also refer to proteins or polypeptides capable of interacting with other cellular or extracellular molecules in a manner substantially similar to the way in which the corresponding portion of the endogenous differentially expressed gene product would. For example, a “functionally equivalent” peptide would be able, in an immunoassay, to diminish the binding of an antibody to the corresponding peptide (i.e., the peptide the amino acid sequence of which was modified to achieve the “functionally equivalent” peptide) of the endogenous protein, or to the endogenous protein itself, where the antibody was raised against the corresponding peptide of the endogenous protein. An equimolar concentration of the functionally equivalent peptide will diminish the aforesaid binding of the corresponding peptide by at least about 5%, preferably between about 5% and 10%, more preferably between about 10% and 25%, even more preferably between about 25% and 50%, and most preferably between about 40% and 50%.

The polypeptides of the present invention may be produced by recombinant DNA technology using techniques well known in the art. Therefore, there is provided a method of producing a polypeptide of the present invention, which method comprises culturing a host cell having incorporated therein an expression vector containing an exogenously-derived polynucleotide encoding a polypeptide comprising an amino acid sequence as set forth in SEQ ID NOs:1, 5 or 6 under conditions sufficient for expression of the polypeptide in the host cell, thereby causing the production of the expressed polypeptide. Optionally, said method further comprises recovering the polypeptide produced by said cell. In a preferred embodiment of such a method, said exogenously-derived polynucleotide encodes a polypeptide consisting of an amino acid sequence set forth in SEQ ID NOs:1, 5 or 6 Preferably, said exogenously-derived polynucleotide comprises the nucleotide sequence as set forth in any of SEQ ID NO:2, SEQ ID NO:3, SEQ ID NO:4, SEQ ID NO: 7 or SEQ ID NO:8. In case of using the nucleotide sequence set forth in SEQ ID NO:3, i.e. the open reading frame, the sequence, when inserted into a vector, may be followed by one or more appropriate translation stop codons, preferably by the natural endogenous stop codon TGA beginning at nucleotide 2021 in the cDNA sequence.

Thus, methods for preparing the polypeptides and peptides of the invention by expressing nucleic acid encoding respective nucleotide sequences are described herein. Methods which are well known to those skilled in the art can be used to construct expression vectors containing protein coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, supra, and Ausubel et al., 1989, supra. Alternatively, RNA capable of encoding differentially expressed gene protein sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

A variety of host-expression vector systems may be utilized to express the HDAC9 gene coding sequences of the invention. Such host-expression systems represent vehicles by which the coding sequences of interest may be produced and subsequently purified, but also represent cells which may, when transformed or transfected with the appropriate nucleotide coding sequences, exhibit the HDAC9 gene protein of the invention in situ. These include but are not limited to microorganisms such as bacteria (e.g., E. coli, B. subtilis) transformed with recombinant bacteriophage DNA, plasmid DNA or cosmid DNA expression vectors containing differentially expressed gene protein coding sequences; yeast (e.g. Saccharomyces, Pichia) transformed with recombinant yeast expression vectors containing the differentially expressed gene protein coding sequences; insect cell systems infected or transfected with recombinant virus expression vectors (e.g., baculovirus) containing the differentially expressed gene protein coding sequences; plant cell systems infected with recombinant virus expression vectors (e.g., cauliflower mosaic virus, CaMV; tobacco mosaic virus, TMV) or transformed with recombinant vectors, including plasmids, (e.g., Ti plasmid) containing protein coding sequences; or mammalian cell systems (e.g. COS, CHO, BlK, 293, 3T3) harboring recombinant expression constructs containing promoters derived from the genonie of mammalian cells (e.g., metallothioneine promoter) or from mammalian viruses (e.g., the adenovirus late promoter; the vaccinia virus 7.5K promoter, or the CMV promoter).

Expression of the HDAC9 of the present invention by a cell from an HDAC9 encoding gene that is native to the cell can also be performed. Methods for such expression are detailed in, e.g., U.S. Pat. Nos. 5,641,670; 5,733,761; 5,968,502; and 5,994,127, all of which are expressly incorporated by reference herein in their entirety. Cells that have been induced to express HDAC9 by the methods of any of U.S. Pat. Nos. 5,641,670; 5,733,761; 5,968,502; and 5,994,127 can be implanted into a desired tissue in a living animal in order to increase the local concentration of HDAC9 in the tissue.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable. In this respect, fusion proteins comprising hexahistidine tags may be used, such as EpiTag vectos including pCDNA3.1/His (Invitrogen, Carlsbad, Calif.). Other vectors include, but are not limited, to the E. coli expression vector pUR278 (Ruther et al., 1983, EMBO J. 2:1791), in which the protein coding sequence may be ligated individually into the vector in flame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety. Fusion proteins containing Flag tags, such as 3X Flag (Sigma, St. Louis, Mo.) or myc tags, for example pCDNA3.1/myc-His (Invitrogen, Carlsbad, Calif.) may be used. These fusions allow coimmunoprecipitation and Western detection of proteins for which antibodies are not yet available.

Promoter regions can be selected from any desired gene using vectors that contain a reporter transcription unit lacking a promoter region, such as a chloramphenicol acetyl transferase (“CAT”), or the luciferase transcription unit, downstream of restriction site or sites for introducing a candidate promoter fragment; i.e., a fragment that may contain a promoter. For example, introduction into the vector of a promoter-containing fragment at the restriction site upstream of the cat gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well known and readily available promoters, but also promoters that readily may be obtained by the foregoing technique, using a reporter gene.

Among known bacterial promoters suitable for expression of polynucleotides and polypeptides in accordance with the present invention are the E. coli lacI and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter. For example, a plasmid construct could contain a HDAC9 transcriptional control sequence fused to a reporter transcription unit that encodes the coding region of β-Galactosidase, chloramphenicol acetyltransferase, green fluorescent protein or luciferase. This construct could be used to screen for small molecules that modulate HDAC9 transcription. Such molecules are potential therapeutics. Furthermore, an HDAC9 reporter gene could be used to examine the effects of an HDAC9 therapeutic in mammalian cells or xenografts using fluorescent reporters and imaging techniques, such as fluorescence microscopy or Biophotonic in vivo imaging, a technology that produces visual and quantitative measurements in real time (Xenogen, Palo Alto, Calif.). Changes in these reporters in normal, diseased or drug-treated tissue or cells would be indicators of changes in HDAC9 expression or activity.

In an insect system, Autographa californica nuclear polyhedrosis virus (AcNPV) is one of several insect systems that can be used as a vector to express foreign genes. The virus grows in Spodoptera frugiperda cells. The coding sequence may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith et al., 1983, J. Virol. 46: 584; Smith, U.S. Pat. No. 4,215,051).

In mammalian host cells, a number of viral-based expression systems may be utilized. In cases where an adenoviris is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the desired protein in infected hosts (e.g., See Logan & Shenk, 1984, Proc. Natl. Acad. Sci. USA 81:3655-3659). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544). Other common systems are based on SV40, retrovirus or adeno-associated virus. Selection of appropriate vectors and promoters for expression in a host cell is a well known procedure and the requisite techniques for expression vector construction, introduction of the vector into the host and expression in the host per se are routine skills in the art. Generally, recombinant expression vectors will include origins of replication, a promoter derived from a highly-expressed gene to direct transcription of a downstream structural sequence, and a selectable marker to permit isolation of vector containing cells after exposure to the vector.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc. and are well known to one of skill in the art.

For long-term, high-yield production of recombinant proteins, stable expression is preferred. For example, cell lines that stably express a differentially expressed protein product of a gene may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the differentially expressed gene protein. Such engineered cell lines may be particularly useful in screening and evaluation of compounds that affect the endogenous activity of the expressed protein.

A number of selection systems may be used, including but not limited to, the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G418 (Colberre-Garapin, et al., 1981, J. Mol. Biol. 150:1); and hygro, which confers resistance to hygromycin (Santerre, et al., 1984, Gene 30:147) genes.

An alternative fusion protein system allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described below, a protein of the present invention may be labeled, either directly or indirectly, to facilitate detection of a complex formed between the protein and a test substance. Any of a variety of suitable labeling systems may be used including, but not limited to, radioisotopes such as ¹²⁵I; enzyme labeling systems that generate a detectable calorimetric signal or light when exposed to substrate; and fluorescent labels.

Where recombinant DNA technology is used to produce a protein of the present invention for such assay systems, it may be advantageous to engineer fusion proteins that can facilitate labeling, immobilization, detection and/or isolation

Indirect labeling involves the use of a protein, such as a labeled antibody, which specifically binds to a polypeptide of the present invention. Such antibodies include but are not limited to polyclonal, monoclonal, chimeric, single chain, Fab fragments and fragments produced by an Fab expression library.

In another embodiment, nucleic acids comprising a sequence encoding HDAC9 protein or functional derivative thereof, may be administered to promote normal biological function, for example, normal transcriptional regulation, by way of gene therapy. Gene therapy refers to therapy performed by the administration of a nucleic acid to a subject. In this embodiment of the invention, the nucleic acid produces its encoded protein that mediates a therapeutic effect by promoting normal transcriptional regulation.

Any of the methods for gene therapy available in the art can be used according to the present invention. Exemplary methods are described below.

In a preferred aspect, the therapeutic comprises a HDAC9 nucleic acid that is part of an expression vector that expresses a HDAC9 protein or fragment or chimeric protein thereof in a suitable host. In particular, such a nucleic acid has a promoter operably linked to the HDAC9 coding region, said promoter being inducible or constitutive, and, optionally, tissue-specific. In another particular embodiment, a nucleic acid molecule is used in which the HDAC9 coding sequences and any other desired sequences are flanked by regions that promote homologous recombination at a desired site in the genome, thus providing for intrachromosomal expression of the HDAC9 nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; and WO 93/20221). Alternatively, the nucleic acid can be introduced intracellularly and incorporated within host cell DNA for expression, by homologous recombination (see, e.g., U.S. Pat. Nos. 5,413,923; 5,416,260; and 5,574,205; and Zijlstra et al., 1989, Nature 342:435-438).

In a specific embodiment, a viral vector that contains the HDAC9 nucleic acid is used. For example, a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The HDAC9 nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular heniatopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a HDAC9 nucleic acid is introduced into the cells such that it is expressible by the cells or their progeny, and the recombinant cells are then administered in vivo for therapeutic effect. In a specific embodiment, stem or progenitor cells are used. Any stem-and/or progenitor cells that can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (see, e.g., WO 94108598), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

A further embodiment of the present invention relates to a purified antibody or a fragment thereof which specifically binds to a polypeptide that comprises the amino acid sequence set forth in SEQ ID NOs:1, 5 or 6 or to a fragment of said polypeptide. A preferred embodiment relates to a fragment of such an antibody, which fragment is an Fab or F(ab′)₂ fragment. In particular, the antibody can be a polyclonal antibody or a monoclonal antibody.

Described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a fingerprint, target, gene in a biological sample, or, alternatively, as a method for the inhibition of abnormal target gene activity. Thus, such antibodies may be utilized as part of disease treatment methods, and/or may be used as part of diagnostic techniques whereby patients may be tested for abnormal levels of the HDAC9 polypeptide, or for the presence of abnormal forms of the HDAC9 polypeptide.

For the production of antibodies to the HDAC9 polypeptide, various host animals may be immunized by injection with the HDAC9 polypeptide, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the HDAC9 polypeptide, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries maybe constructed (Huse et al., 1989, Science, 246:1275-1281) to allow rapid and easy identification of monoclonal Fab fragments with the desired specificity.

An antibody of the present invention can be preferably used in a method for the diagnosis of a condition associated with abnormal HDAC9 expression or activity, for example, abnormal cell proliferation, cancer, atherosclerosis, inflammatory bowel disease, host inflammatory or immune response, or psoriasis, in a human which comprises: measuring the amount of a polypeptide comprising the amino acid sequence set forth in SEQ ID NOs:1, 5 or 6, or fragments thereof, in an appropriate tissue or cell from a human suffering from a condition associated with abnormal HDAC9 activity, wherein the presence of an elevated amount of said polypeptide or fragments thereof, relative to the amount of said polypeptide or fragments thereof in the respective tissue from a human not suffering from a condition associated with abnormal HDAC9 activity is diagnostic of said human's suffering from such condition. Such a method forms a further embodiment of the present invention. Preferably, said detecting step comprises contacting said appropriate tissue or cell with an antibody which specifically binds to a polypeptide that comprises the amino acid sequence set forth in SEQ ID NOs:1, 5 or 6 or a fragment thereof and detecting specific binding of said antibody with a polypeptide in said appropriate tissue or cell, wherein detection of specific binding to a polypeptide indicates the presence of a polypeptide that comprises the amino acid sequence set forth in SEQ ID NOs:1, 5 or 6 or a fragment thereof.

Particularly preferred, for ease of detection, is the sandwich assay, of which a number of variations exist, all of which are intended to be encompassed by the present invention.

For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the HDAC9 polypeptide or a fragment thereof.

The most commonly used reporter molecules in this type of assay are either enzymes, fluorophore- or radionuclide-containing molecules. In the case of an enzyme inmmunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon, hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of HDAC9 which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

This invention also relates to the use of polynucleotides of the present invention as diagnostic reagents. In particular, the invention relates to a method for the diagnosis of a condition associated with abnormal HDAC9 expression or activity, for example, abnormal cell proliferation, cancer, atherosclerosis, inflammatory bowel disease, host inflammatory or immune response, or psoriasis in a human which comprises:detecting elevated transcription of messenger RNA transcribed from the natural endogeneous human gene encoding the polypeptide consisting of an amino acid sequence set forth in SEQ ID NOs:1, 5 or 6 in an appropriate tissue or cell from a human, wherein said elevated transcription is diagnostic of said human's suffering from the condition associated with abnormal HDAC9 expression or activity. In particular, said natural endogeneous human gene comprises the nucleotide sequence set forth in SEQ ID NO:4. 7 or 8. In a preferred embodiment such a method comprises contacting a sample of said appropriate tissue or cell or contacting an isolated RNA or DNA molecule derived from that tissue or cell with an isolated nucleotide sequence of at least about 20 nucleotides in length that hybridizes under high stringency conditions with the isolated nucleotide sequence encoding a polypeptide consisting of an amino acid sequence set forth in SEQ ID NOs:1, 5 or 6.

Detection of a mutated form of the gene characterized by the polynucleotide of SEQ ID NO:4 7 or 8 which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or CDNA may also be used in similar fashion. Deletions and insertions can be detected by a change in size of the amplified product in comparison to the normal genotype. Point mutations can be identified by hybridizing amplified DNA to labeled nucleotide sequences encoding the HDAC9 polypeptide of the present invention. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (e.g., Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401). In another embodiment, an array of oligonucleotides probes comprising nucleotide sequence encoding the HDAC9 polypeptide of the present invention or fragments of such a nucleotide seqeunce can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to disease through detection of mutation in the HDAC9 gene by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit which comprises:

(a) a polynucleotide of the present invention, preferably the nucleotide sequence of SEQ ID NO:2, 3, 4, 7 or 8 or a fragment thereof;

(b) a nucleotide sequence complementary to that of (a);

(c) a polypeptide of the present invention, preferably the polypeptide of SEQ ID NOs:1, 5 or 6 or a fragment thereof; or

(d) an antibody to a polypeptide of the present invention, preferably to the polypeptide of SEQ ID NOs:1, 5 or 6.

It will be appreciated that in any such kit, (a), (b), (c) or (d) may comprise a substantial component. Such a kit will be of use in diagnosing a disease or susceptibility to a disease, particularly to a disease or condition associated with abnormal HDAC9 expression or activity, for example, abnormal cell proliferation, cancer, atherosclerosis, inflammatory bowel disease, host inflammatory or immune response, or psoriasis.

The nucleotide sequences of the present invention are also valuable for chromosome localization. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

An additional embodiment of the invention relates to the administration of a pharmaceutical composition, in conjunction with a pharmaceutically acceptable carrier, excipient or diluent, for any of the therapeutic effects discussed above. Such pharmaceutical compositions may consist of HDAC9, antibodies to that polypeptide, mimetics, agonists, antagonists, or inhibitors of HDAC9 function. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

In addition, any of the therapeutic proteins, antagonists, antibodies, agonists, antisense sequences or vectors described above may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects. Antagonists and agonists of HDAC9 may be made using methods which are generally known in the art.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated m aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of the HDAC9, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example HDAC9 or fragments thereof, antibodies of HDAC9, agonists, antagonists or inhibitors of HDAC9, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The following Examples illustrate the present invention, without in any way limiting the scope thereof.

### EXAMPLES

### Example 1

### Identification of a Novel HDAC Related Human DNA Sequence Using Bioinformatics

HDAC9 was identified using computer software for the identification of new members of gene families based on a strategy to find maximal evolutionary links among known HDAC family members by first searching the non-redundant amino acid database, followed by searching less diverse databases such as the Celera Human Genome Database (CHGD), public High Throughput Genomic (HTG) database and the Incyte LIFESEQ™ database. Smith-Waterman (Pearson W. R. Comparison of methods for searching protein sequence databases. Protein Sci (1995) 4,1145-60) and Hidden Markov Models (probability models derived from diversity of amino acids at every position (Eddy S. R. Hidden Markov models. Curr Opin Struct Biol (1996) 6, 361-5) were performed. An 1156 bp open reading frame (ORF) was identified and used to search a database of sequenced clones from pan-tissue and dorsal root ganglion cDNA libraries.

### Example 2

### Construction of Pan-tissue and Dorsal Root Ganglion cDNA Libraries

Pan-tissue and dorsal root ganglion cDNA libraries are prepared from polyA+RNA. Total RNA was extracted from a pooled sample of 31 human tissues or dorsal root ganglia and isolated using TRIZOL reagent according to manufacturer's instructions (Life Technologies, Rockville, Md.). mRNA is isolated using Polytract mRNA Isolation System III according to manufacturer's instructions (Promega, Madison, Wis.). Total RNA is hybridized to a biotinylated-oligo (dt) probe. The oligo (dt)-mRNA hybrids are captured on streptavidin magnesphere particles and eluted in Rnase-free H₂0. 3 ul of biotinylated-oligo(dt) probe (50 pmol/ul) and 13 ul of 20×SSC is added to 60-150 ug of RNA that is heated to 65° C. in RNase free water. This mixture is incubated at room temperature until it is completely cooled. Streptavidin-paramagnetic particles (beads) are resuspended and washed 3 times in 0.5×SSC and then resuspended in 0.5×SSC. The RNA-oligo(dt) hybrids from the previous step are added to these beads. To release the poly-A RNA from the beads, the beads are resuspended in Rnase-free water and magnetically captured and then the eluate from the beads is ethanol precipitated. First and second strand cDNA synthesis is performed using a modified procedure from Life Technologies (D'Alessio, J. M., Gruber, C. E., Cain, C. R., and Noon, M. C. (1990) Focus 12, 47). First strand synthesis is performed by incubating 1-5 ug of RNA that is heated to 60° C. in 1× 1^{st} strand buffer (Life Technologies)/6 mM DTT/600 nM dNTPs/2 units anti-Rnase. This mixture is incubated at 40° C. for2 min, then Superscript II reverse transcriptase (RT) and 1 ul of Display Thermo RT terminator mix is added and the mixture is incubated at 40° C. for 1 h, followed by incubation at 60° C. for 10 min. Second strand synthesis is performed in 1× second strand buffer (Life Technologies) in DEPC-H₂0/66 nM/1 ul E.coli DNA ligase/4 ul E. coli DNA polymerase I/1 ul E. coli Rnase H. This mixture is incubated at 10° C. for 10 min and then at 16° C. for 2 h. To this mixture, 2 ul of T4 DNA polymerase is added and incubation is continued at 16° C. for 5 min. The reaction is stopped with 10 ul of 0.5M EDTA, extracted with pheno/chloroform/isoamyl alcohol and then ethanol precipitated. Sal I and Not I adaptors are added to the 5′ ends of the cDNAs by ligation for directional cloning using conventional methodology. The cDNAs are then passed through a size fractionation column to retrieve cDNAs that are >500 bp in length according to manufacturers instructions (Life Technologies, Rockville, Md.). cDNAs are ligated to Sal I/Not I digested Gateway compatible pCMV-Sport6 vector (Life Technologies, Rockville, Md.) using conventional methods. Competent DH10B cells (Life Technologies, Rockville, Md.) are transformed with the resulting library using conventional methods. Semi-solid amplification of the libraries is performed according to the manufacturer's instructions (Life Technologies, Rockville, Md.).

### Example 3

### Preparation of Full Length cDNA Encoding the Novel HDAC9 Consisting of SEQ ID NO:1, 5 or 6

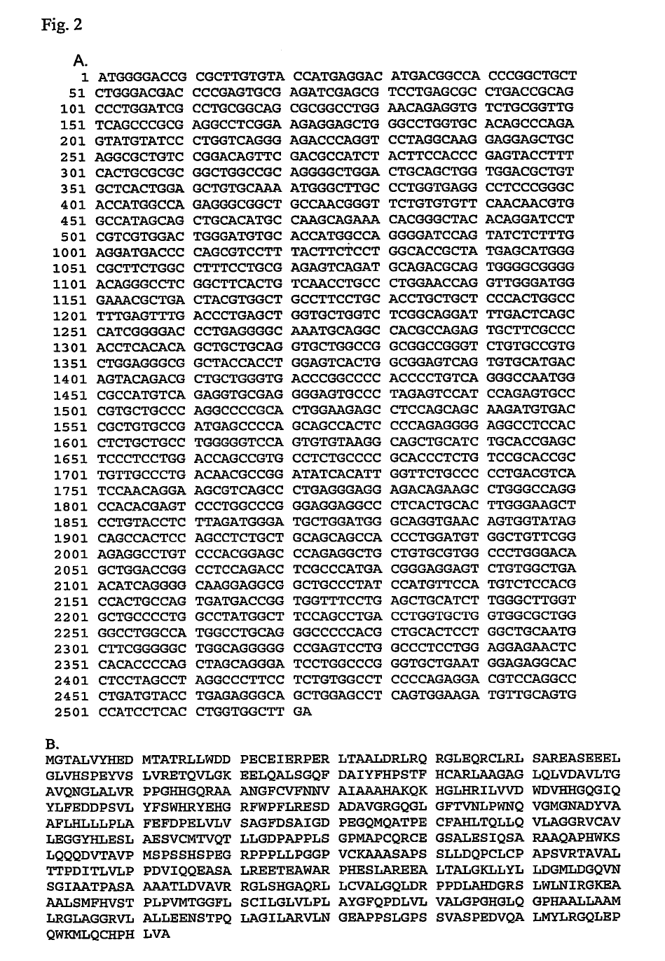

An 1156 base pair ORF was used to search a database of sequenced clones from pan-tissue and dorsal root ganglion cDNA libraries using BLAST. Four clones were found to contain the ORF (M6, K10, P3, F23), two from each library. Of these clones M6 from the pan-tissue library was determined to be the most complete, but missing approximately 44 bp from the N-terminus. A protein slightly smaller than that predicted for the complete cDNA was observed by in vitro translation. The result that proteins were observed by in vitro translation of the incomplete cDNA, suggests possibility of alternate translation initiation sites within HDAC9. Specifically, sequencing of HDAC9 in pCMVSport6 was performed using an automated ABI Sequencer (ACGT, Northbrook, Ill.). PCR was performed using conditions listed in the ABI Prism BigDye™ Terminator Cycle Sequencing Ready Reaction Kit manual and are as follows: denaturation at 96° C. for 30 seconds, annealing at 50° C. for 15 seconds, extension at 60° C. for 4 minutes, for a total of 25 cycles. Each round of sequencing provided between 200 and 600 bp of sequence. PCR primers for 1^{st} round sequencing were 5′-ATTTAGGTGACACTATAG-3′ (Sp6, sense) and 5′-TAATACGACTCACTATAGGG-3′ (T7, antisense). Results of sequencing using Sp6 primer are as follows. Bolded sequence is pCMVSport6 vector sequence. CTggtACCGGTCCGGAATTCCCGGGATATCGTCGACCCACGCGTCCG/GGCTGCT CCCGGCCGAAGCCCCGAGTGCGAGATCGAGCGTCCTGAGCGCCTGACCGCAGCCCT GGATCGCCTGCGGCAGCGCGGCCTGGAACAGAGGTGTCTGCGGTTGTCAGCCCGCG AGGCCTCGGAAGAGGAGCTGGGCCTGGTGCACAGCCCAGAGTATGTATCCCTGGTC AGGGAGACCCAGGTCCTAGGCAAGGAGGAGCTGCAGGCGCTGTCCGGACAGTTCGA CGCCATCTACTTCCACCCGAGTACCTTICACTGCGCGCGGCTGGCCGCAGGGGCTGG ACTGCAGCTGGTGGACGCTGTGCTCACTGGAGCTGTGCA:AAATGGGCTTGCCCTGG TGAGGCCTCCCGGGCACCATGGCCAGAGGGCGGCTGCAACGGGTTCTGCGTGTTCA ACAACGTGGCCATAGCAGCTGCACATGCCAAGCAGAAACACGGGCTACACAGGATC CTCGTCGTGGACTGGGGGATGTGCACCATGGCAGGGGGATCCAGTATCTCTTTGAAG GATGACCCCAGCGTCCTTTACTTCTCCTGGCACCGCTATGAGCATTGGGCGCCTTCT GGCCITTCTGCGAGAGTCAGATGAgACGCATGGGGGGCGGGGGACAGGGCCTCGGC TTCACTGTCaACCTGCCCTGACCAAGTTgGGGGAATGGGGAAACGCTGACTTACGTG GCTGGCCTTCTTGCACCTTGCTGGTTCCAcTGGCCTTTTGGAGTTTGACCTGAgCTGG GTGCTTGGTcTCgGCAGGGATTTGACTcagcCaTtCgGGACCCTGAgGGGGCAAA. Results of sequencing using the T7 primer were: TCAAGCCACCAGGTGAGGATGGCACTGCAACATCTTCCACTGAGGCTCCAGCTGCCC TCTCAGGTACATCAGGGCCTGGACGTCCTCTGGGGAGGCCACAGAGGAAGGGCCTA GGCTAGGAGGTGCCTCTCCATTCAGCACCCGGGCCAGGATCCCTGCTAGCTGGGGTG TGGAGTTCTCCTCCAGGAGGGCCAGGACTCGGCCCCCTGCCAGCCCCCGAAGCATTG CAGCCAGGAGTGCAGCGTGGGGGCCCTGCAGGCCATGGCCAGGCCCCAGCGCCACC AGCACCAGGTCAGGCTGGAAGCCATAGGCCAGGGGCAGCaCCAAGCCCAAGATGCA GCTCAGGAAACCACCGGTCATCACTGGCAGTGGCGTGGAGACATGGAACATGGA[T AGGGCAGcCGCCTCCTTGCCCTGATGTTCAGCCACAGACTcCTCCCGTCATGGGCGA AGTCTGGAGGCCGGTCCAgCTGTtaGGCCACGCACAGAgtCTCTGGGCTCCgtGGGACA gGCCT:TTTtGAAAAGAtATTtAGGGTGGGTTGTGAacaggGCTGGAATGGCTGGTATAcC AcTGtTTAcCTGCCATT. 2^{nd} and 3^{rd} round sequencing primers are designed to prime sequence obtained from the previous round of sequencing. 2^{nd} round primers are 5′-GTCATCA CTGGCAGTGGCGTG-3′ (HUF7392, antisense) and 5′-TGGACTGCAGCTGGTGG-3′ (DF-2, sense). Results of sequencing using the DF-2 primer were: CTGGcAAATGGGCTTGCCCTGG TGAGGCCTCCCGGGCACCATGGCCAGAGGGCGGCTGCCAACGGGTTCTGCGTGTTC AACAACGTGGCCATAGCAGCTGCACATGCCAAGCAGAAACACGGGCTACACAGGAT CCTCGTCGTGGACTGGGATGTGCACCATGGCCAGGGGATCCAGTATCTCTITGAGGA TGACCCCAGCGTCCTTTACTTCTCCTGGCACCGCTATGAGCATGGGCGCTTCTGGCCT TTCCTGCGAGAGTCAGATGCAGACGCAGTGGGGCGGGGACAGGGCCTCGGCTTCAC TGTCAACCTGCCCTGGAACCAGGTTGGGATGGGAAACGCTGACTACGTGGCTGCCTT cCTGCACCTGCTGCTCCCACTGGCCTTTGAGTTTGACCCTGAGCTGGTGCTGGTCTCG GCAGGATTTGACTCAGCCATCGGGGACCCTGAGGGGCAAATGCAGGCCACGCCAGA GTGCTTCGCCCACCTCACACAGCTGCTGCAGGTGCTGGCCGGCGGCCGGGTCTGTGC CGTGCTGGAGGGCGGCTACCACCTGGAGTCACTGGCGGAGTCAGTGTGCATGACAG TACAGACGCTGCTGGGTGACCCGGcCCCACCCCTGTCAGGGCCAATGGCGCCATGTC AGAGTGCCCTAgAgTCATTCAgAGTGCCCGTGCTGCCAGGcCCCGCACTGGAAAgAgG CTTCAgCAGCAAgATGTGACCGcTGTGCCGATGAACCCCA. Sequencing results for the HUF7392 primer were: TGtaTAGGGcAGCCGCCTCCTTGCC CCTGATGTTCAGCCACAGACTCCTCCCGTCATGGGCGAGG TCTGGAGGCCGGTCCAGCTGTCCCAGGGCCACGCACAGCAGCCTCTGGGCTCCGTG GGACAGGCCTCTCCGAACAGCCACATCCAGGGTGGCTGCTGCAGCAGAGGCTGGAG TGGCTGCTATACCACTGTTCACCTGCCCATCCAGCATCCCATCTAAGAGGTACAGGA GCTTCCCAAGTGCAGTGAGGGCCTCCTCCCGGGCCAGGGACTCGTGTGGCCTGGCCC AGGTCTTCTGTCTCCTCCCTCAGGGCTGACGCTTCTGTTGGATGACGTCAGGGGGCAG AACCAATGTGATATCCGGCGTTGTCAAGGGCAACAGCGGTGCGGACAGAGGGTGCG GGGCAGAGGCACgGCTGGTCCAgGAGGGAGCTCGGTGCAgATGCAGcTGCCTTACAC ACTGgACCCCCAGGCAGCAGAGGTGGAGGCCTCCCCTCTGGGGAGTG. 3^{rd} round sequencing primers were 5′-AACAGCGGTG C GGACAGA-3′ (HUF2A, antisense) and 5′-CTGGAGTCACTGGCGGAG-3′ (DF3A, sense). Results of sequencing using DF3A primer were: AgcaCAGA cGCTgCTGGGTGACCCGGCCCACCCCTG TCAGGGCCAATGGCGCCATGTCAGAGTGCCCTAGAGTCCATCCAGAGTGcCCGTGCT GCCCAGGCCCCGCACTGGAAGAGCCTCCAGCAGCAAGATGTGACCGCTGTGCCGAT GAGCCCCAGCAGCCACTCCCCAGAGGGGAGGCCTCCACCTCTGCTGCCTGGGGGTC CAGTGTGTAAGGCAGCTGCATCTGCACCGAGCTCCCTCCTGGACCAGCCGTGCCTCT GCCCCGCACCCTCTGTCCGCACCGCTGTTGCCCTGACAACGCCGGATATCACATTGG TTCTGCCCCCTGACGTCATCCAACAGGAAGCGTCAGCCCTGAGGGAGGAGACAGAA GCCTGGGCCAGGCCACACGAGTCCCTGGCCCGGGAGGAGGCCCTcACTGcACTTGGG AAGCTCCTGTACCTcTTAgATGGGATGCTGGATGGGCAGGTGAACAgTGGTATA. Results of sequencing using HUF2A primer were: TgcaCGGATGGTCCAGGAGGGAGCTCG GTGCAAATGCAGCTGCCTTACACACTGGACCCCCAGGCAGCAgAGGTGGAGGCCTC CCCTcTGGGGAGTGGCTGCTGGGGCTCATCGGCACAGCGGTCACATCTrGCTGCTGG AGGCTCTTCCAGTGCGGGGCCTGGGCAGCACGGGCACTCTGGATGGACTCTAGGGC ACTCTGACATGGCGCCATTGGCCCTGACAGGGGTGGGGCCGGGTCACCCAGCAGCG TCTGTACTGTCATGCACACTGACTCCGCCAGTGACTCCAGGTGGTAGCCGCCCTCCA GCACGGCACAgACCCGGCCGCCGGCCAGCACCTGCAGCAGCTGTGTGAGGTGGGCg AAGCACTCTGGCGTGGCCTGCATTTGCCCCTCAGGGTCCCCGATGGCTTGAGTCAAA TCCTGCCGAGACCAGCACCAGCTCAGGGTCAAACTCAAAGGCCAGTGGGAGCAGCA GGTGCAGGAAGGCAGCCACgTATCAGCGTTTCCCATCCCAACCTGgTTCCAGGGGCA GGTTGAACAGTGAAGCCGAGGGCCCCTTGTCCCCgCCCCACCTTGCGTCTGCATctGA CTCTCGCAGGAAAGGCCAAgAAGCgCCCATgCTATTTT. The overlapping sequence from the combined sense and antisense sequencing was reconstructed to give the complete cDNA sequence of HDAC9. See FIG. 2A.

BLAST is used to search the Genbank database using cDNA clone M6 as the query to identify a genomic sequence containing M6 cDNA sequence. The results of this search identified a genomic sequence AL022328 that was found to contain exons that were identical in sequence to the M6 cDNA. The sequence of cDNA clone M6 was confirmed by automated DNA sequencing (ACGT, Inc. Northbrook, Ill.). See FIG. 2A.

The remaining 44 bp of N-terminal sequence was added by PCR using the nested sense strand primers 5′-GCGGTCGACGCCACCATGGGGACCGCGCTTGTGTACCATGAGGAC ATG-3′ and 5′-GTGTACCATGAGGACATGACGGCCACCCGGCTGCTCTGGGACGACC CCGAGTGC-3′ and the 3′ primer 5′-GAACCAATGTGATATCCGGCGTTG-3′. The 5′-primer added a kozak sequence and a Sal1 site for cloning and the 3′ primer sequence overlaps the EcoRV site in HDAC9. PCR was performed using a step-cycle file for amplification using 1 cycle of 94° C. for 30 seconds, 68° C. for 30 seconds, and 72° C. for 1 minute, followed by 20 cycles of 94° C. for 30 seconds and 72° C. for 1 minute.

### Example 3

### HDAC9 Sequence Variants

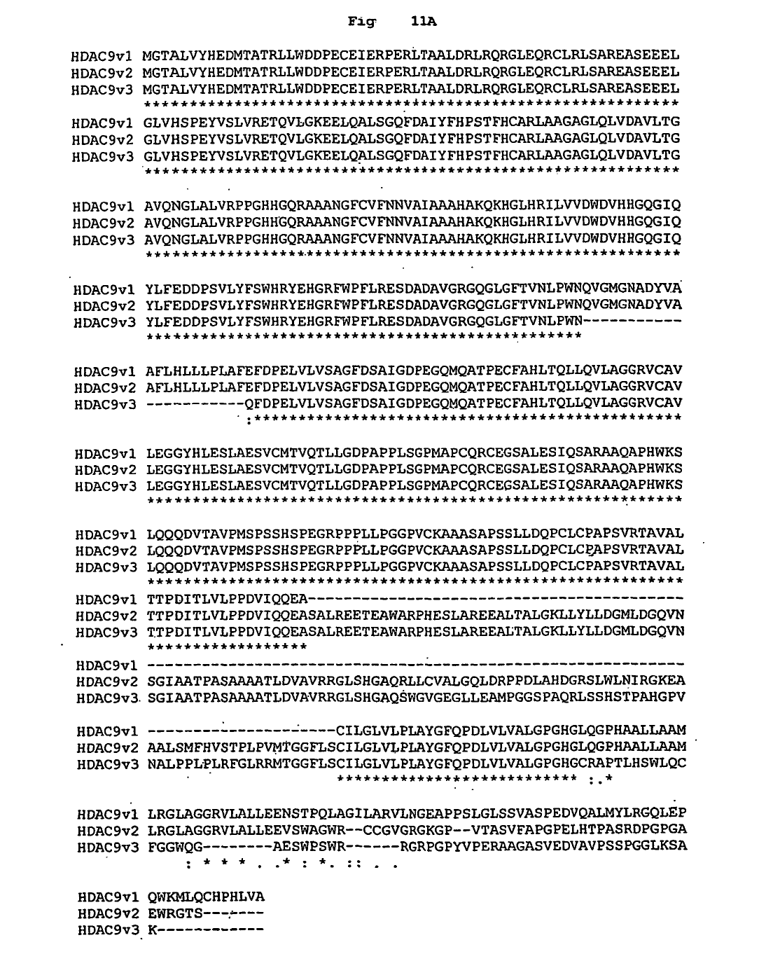

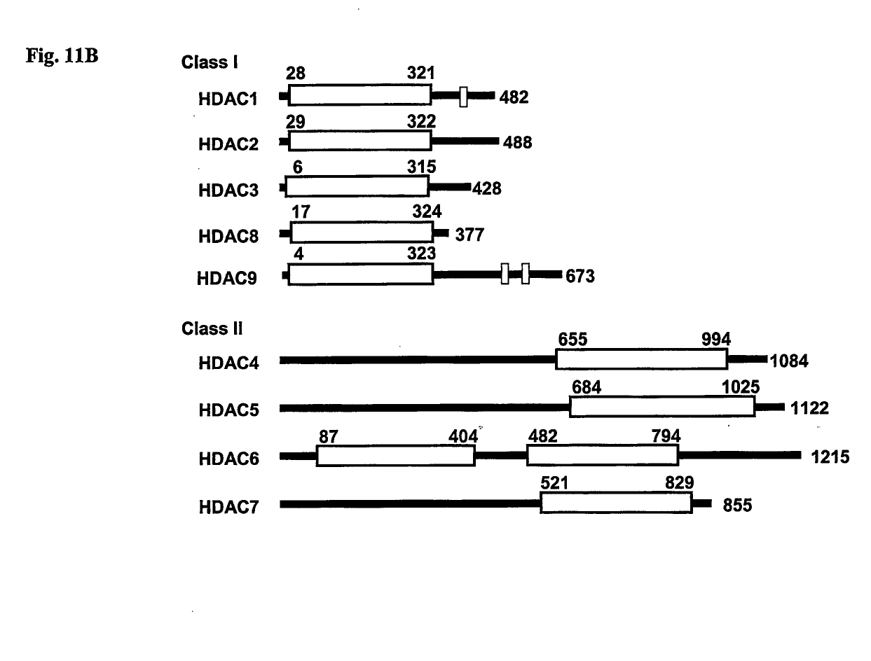

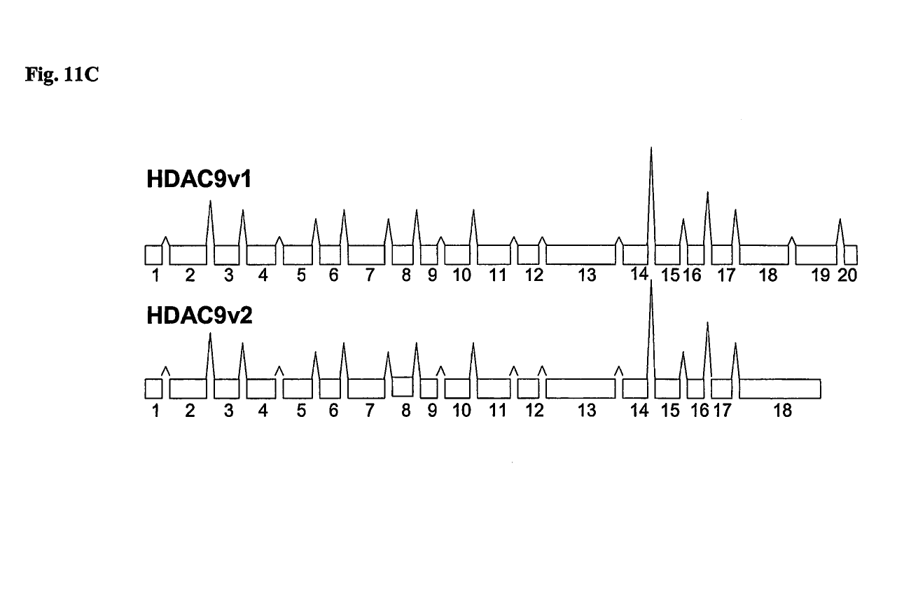

Three variants of the HDAC9 sequence, HDAC9v1, HDAC9v2, and HDAC9v3 were found. HDAC9v1 is the original sequence found and described above. HDAC9v2 was found in the human dorsal root ganglion cDNA library and in AL022328 genomic sequence. HDAC9v3 is a predicited transcript that lacks a stop codon that was found in the Celera human genomic database. HDAC9v1 contains 20 exons and HDAc9v2 has 20 exons. Comparison of the peptide sequences of HDAC9 variants demonstrated that HDAC9v1 and HDAC9v2 were identical up to exon 17, but diverge after this exon. HDAC9v2 has an extended intron between exon 17 and 18 and an extended exon 18 that contains HDAC9v1 exon 19, but lacks 20, as a result of a single nucleotide insertion at nucleotide 446. This insertion frame shifts the sequence and shortens the peptide by 11 amino acids (FIG. 11A). Compared to HDAC9v1 and HDAC9v2, HDAC9v3 has an internal deletion of amino acids 219 through 240 and diverges in its C-terminal beginning at amino acid 486. HDAC9 is the first HDAC enzyme for which sequence variants have been reported. HDAC9v1 is the sequence variant that is characterized, unless otherwise noted.

### Example: 4

### Identification of HDAC-associated Sequence Motifs

The M6 clone was analyzed for the presence of motifs that would indicate an HDAC catalytic domain and a binding site for Rb and Rb-like proteins. HDACs are characterized by the presence of a catalytic domain with conserved amino acids. Most of the HDACs that have been identified to date have one catalytic domain, with the exception of HDAC6 that has two domains. N-terminal catalytic domains have been associated with class I HDACs, while C-terminal catalytic domains are associated with class II HDACs. An N-terminal catalytic domain was found in HDAC9 based upon PFAM prediction and alignment with the catalytic domains of other HDACs. A set of conserved amino acids were previously shown to be critical for HDAC activity and provide the critical contacts for HDAC inhibitor, TSA, based upon single amino acid mutations in HDAC1 and the three dimensional structure formed by a complex of an HDAC-like protein (HDLP), Zn²⁺ and HDAC inhibitor TSA (Hassig C A, Tong J K, Fleischer T C, Owa T, Grable P G, Ayer D E, Schreiber S L. (1998) Proc Natl Acad Sci USA. 95, 3519-3524; Finnin, M. S., Doniglan, J. R, Cohen, A., Richon, V. M., Rifkind, R. a, Marks, P. A., Breslow, R., and Pavletich, N. P. (1999) Structures of a histone deacetylase homologue bound to TSA and SAHA inhibitors. Nature 401, 188-193). A bacterial protein with similarities in sequence and enzymatic activity to human HDACs and the only class I HDAC-like structure elucidated, HDLP was used as an HDAC template. Many of these conserved amino acids with a few exceptions were found in HDAC9 (Table 4). Alignments of HDAC peptide sequences indicated that the hydrophobic residue Leu 265 that forms part of the binding pocket in HDLP is replaced with Glu at amino acid 272 in HDAC9. Similarly, Leu 265 is also replaced with Met in HDAC8 and with Lys in HDAC6 domain 1. Furthermore, Asp 173 in HDLP is substituted with Gln at position 177 in HDAC9, a difference that was also found in the HDAC6 catalytic domain 1. This Asp is substituted with Asn in HDAC4, HDAC5, HDAC6 domain 2, and HDAC7. HDAC1-8 have been shown to be catalytically active, hence the amino acid substitutions in these proteins have no enzymatic consequences.

HDAC9 is similar in sequence to class I and class II HDACs. HDACs have been classified by their sequence similarity with yeast HDACs Rpd3, Hda1, and Sir2 and by catalytic domain location. Alignment of the peptide sequences of HDAC9, yeast HDACs Rpd3, Hda1, Hda1 subfamily member from fission yeast, cryptic loci regulator 3 (Clr3), and Sir2 determined that HDAC9 had the highest sequence similarity with Clr3 (Table 1). However, the sequence similarity is not high enough to categorize HDAC9.

Alignment of human HDACs 1-9 and Sir 1-7 peptide sequences demonstrated that HDAC9 was most similar to class II human HDAC6 (Table 2). Alignment of class I and class II HDAC catalytic domains with HDAC9 catalytic domains demonstrated that HDAC6 catalytic domain 1 has the most sequence similarity with HDAC9 (Table 3).

In order to compare the locations of catalytic domains in HDACs, PFAM predictions were made of the catalytic domains in HDAC peptides (FIG. 11B). The location of HDAC9 catalytic domain was at the N-terminus, similar to class I HDACs, and was estimated as spanning the amino acid sequence from amino acid 4 to 323. In addition, the average length of class I HDACs is 443 amino acids, while the average length of class II HDACs is 1069 amino acids. The 673 amino acid HDAC9 peptide is between the average sizes of class I and class II HDACs (FIG. 11B).

TABLE 1

| HDAC | HDAC | % Similarity to |
|---|---|---|
| Class | Isoform | HDAC9 |
| Class I | Rpd3 | 16 |
| Class II | Hda1 | 18 |
| | Clr3 | 23 |
| Class III | Sir2 | 5 |

TABLE 2

| HDAC | HDAC | % Similarity to |
|---|---|---|
| Class | Isoform | HDAC9 |
| Class I | HDAC1 | 14 |
| | HDAC2 | 15 |
| | HDAC3 | 15 |
| | HDAC8 | 22 |
| Class II | HDAC4 | 21 |
| | HDAC5 | 19 |
| | HDAC6 | 37 |
| | HDAC7 | 20 |
| Class III | Sir1 | 5 |
| | Sir2 | 7 |
| | Sir3 | 11 |
| | Sir4 | 4 |
| | Sir5 | 8 |
| | Sir6 | 10 |
| | Sir7 | 15 |

TABLE 3

| HDAC | HDAC | % Similarity to |
|---|---|---|
| Class | Isoform | HDAC9 |
| Class I | HDAC1 | 20 |
| | HDAC2 | 20 |
| | HDAC3 | 20 |
| | HDAC8 | 19 |
| Class II | HDAC4 | 39 |
| | HDAC5 | 38 |
| | HDAC6-1 | 55 |
| | HDAC6-2 | 53 |
| | HDAC7 | 40 |

The protein product of the retinoblastoma protein (Rb) gene is a transcriptional regulator that controls DNA synthesis, the cell cycle, differentiation and apoptosis and plays a tissue-specific role normal development. Rb complexes with the transcription factor E2F, an interaction that is regulated by phosphorylation. Mutations in Rb lead to a hereditary form of cancer of the retina, retinoblastoma. Mutations have also been found in a number of mesenchymal and epithelial cancers. Mutations that affect regulators of Rb phosphorylation including, cyclin D1, cdk4, and p16 have been found in many cancers. Therefore, Rb function is thought to play a critical role in tumorigenesis (Sellers, W. R., Kaelin, W. G. Jr. (1997) J. Clin. Oncol. 15, 3301-3312, DiCiommo, D., Gallie, B. L., Bremner, R.(2000) Semin. Cancer Biol. 10, 255-269). An Rb-binding motif was previously defined as the amino acid sequence LXCXE, where “X” can be any amino acid (Chen, T. -T. and Wang, J. Y. J. (2000) Mol. Cell Biol. 20, 5571-5580). The LXCXE domain in HDAC1 was found to be dispensible for growth suppression function of Rb, but necessary for HDAC binding to Rb. Two putative Rb-binding motifs were found in HDAC9 (FIG. 11A, green boxes). LLCVA is located between amino acids 510 and 515, and LSCIL located between amino acids 560 and 564. Both are present in HDAC9v1 and HDAC9v2.

TABLE 4

| HDAC | | | | | | | | | | | | | | |
|---|---|---|---|---|---|---|---|---|---|---|---|---|---|---|
| Isoform | Amino acids in the catalytic domains of HDAC isoforms | | | | | | | | | | | | | |
| HDLP | Pro | Tyr | His | His | Gly | Phe | Asp | Asp | His | Asp | Phe | Asp | Leu | Tyr |
| | 22 | 91 | 131 | 132 | 140 | 141 | 166 | 168 | 170 | 173 | 198 | 258 | 265 | 297 |
| HDAC1 | Pro | Glu | His | His | Gly | Phe | Asp | Asp | His | Asp | Phe | Asp | Leu | Tyr |
| | 29 | 98 | 139 | 140 | 148 | 149 | 174 | 176 | 178 | 181 | 205 | 264 | 271 | 303 |
| HDAC2 | Pro | Glu | His | His | Gly | Phe | Asp | Asp | His | Asp | Phe | Asp | Leu | Tyr |
| | 30 | 99 | 140 | 141 | 149 | 150 | 175 | 177 | 179 | 182 | 206 | 265 | 272 | 304 |
| HDAC3 | Pro | Asp | His | His | Gly | Phe | Asp | Asp | His | Asp | Phe | Asp | Leu | Tyr |
| | 23 | 92 | 134 | 135 | 143 | 144 | 167 | 168 | 171 | 174 | 199 | 259 | 266 | 298 |
| HDAC4 | Pro | Trp | His | His | Gly | Phe | Asp | Asp | His | Asn | Phe | Asp | Leu | His |
| | 592 | 762 | 802 | 803 | 900 | 901 | 838 | 839 | 843 | 846 | 870 | 934 | 943 | 976 |
| HDAC5 | Pro | Trp | His | His | Gly | Phe | Asp | Asp | His | Asn | Phe | Asp | Leu | His |
| | 705 | 793 | 832 | 833 | 841 | 842 | 868 | 869 | 873 | 876 | 900 | 964 | 973 | 1006 |
| HDAC6- | Pro | Tyr | His | His | Gly | Tyr | Asp | Asp | His | Gln | Phe | Asp | Lys | Tyr |
| 1 | 106 | 175 | 215 | 216 | 224 | 225 | 251 | 252 | 255 | 258 | 283 | 946 | 353 | 386 |
| HDAC6- | Pro | Tyr | His | His | Gly | Phe | Asp | Asp | His | Asn | Phe | Asp | Leu | Tyr |
| 2 | 501 | 570 | 593 | 594 | 602 | 603 | 647 | 648 | 651 | 654 | 679 | 742 | 749 | 782 |
| HDAC7 | Pro | Tyr | His | His | Gly | Phe | Asp | Asp | His | Asn | Phe | Asp | Leu | His |
| | 502 | 589 | 629 | 630 | 638 | 639 | 668 | 669 | 673 | 676 | 700 | 764 | 773 | 806 |
| HDAC8 | N/A | Tyr | His | His | Gly | Phe | Asp | Asp | His | Asp | Phe | Asp | Met | Tyr |
| | | 100 | 141 | 142 | 150 | 151 | 176 | 177 | 180 | 183 | 208 | 267 | 274 | 306 |
| HDAC9 | Pro | Tyr | His | His | Gly | Phe | Asp | Asp | His | Gln | Phe | Asp | Glu | Tyr |
| | 21 | 94 | 134 | 135 | 143 | 144 | 170 | 173 | 174 | 177 | 205 | 265 | 272 | 305 |

Non-conserved amino acids (bold text).
No alignment (N/A)

### Example 5

### mRNA Distribution of HDAC9 in Normal Tissues

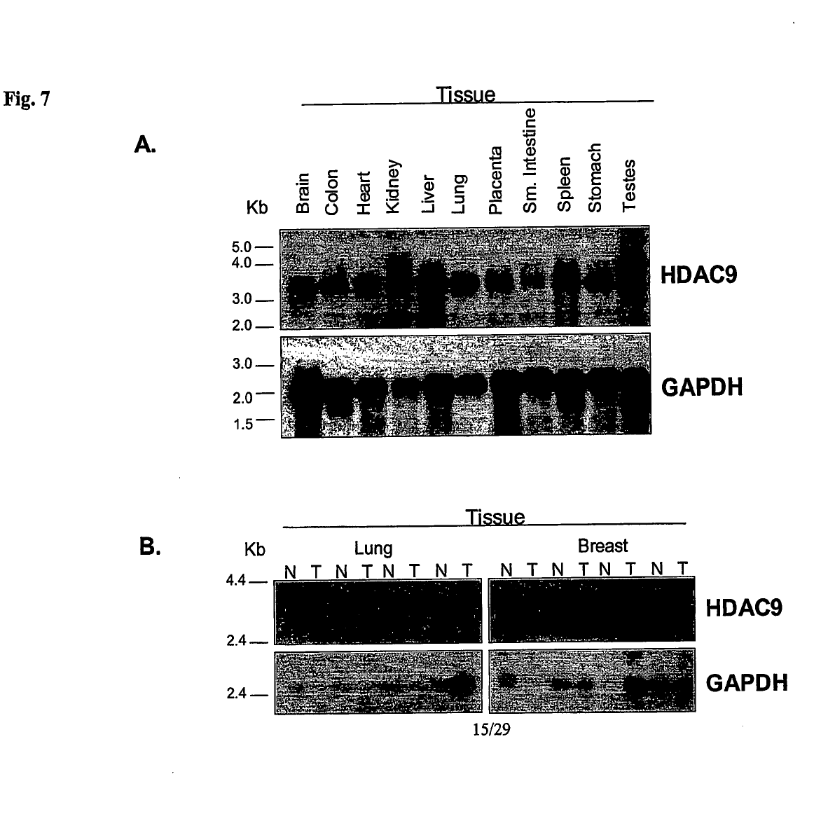

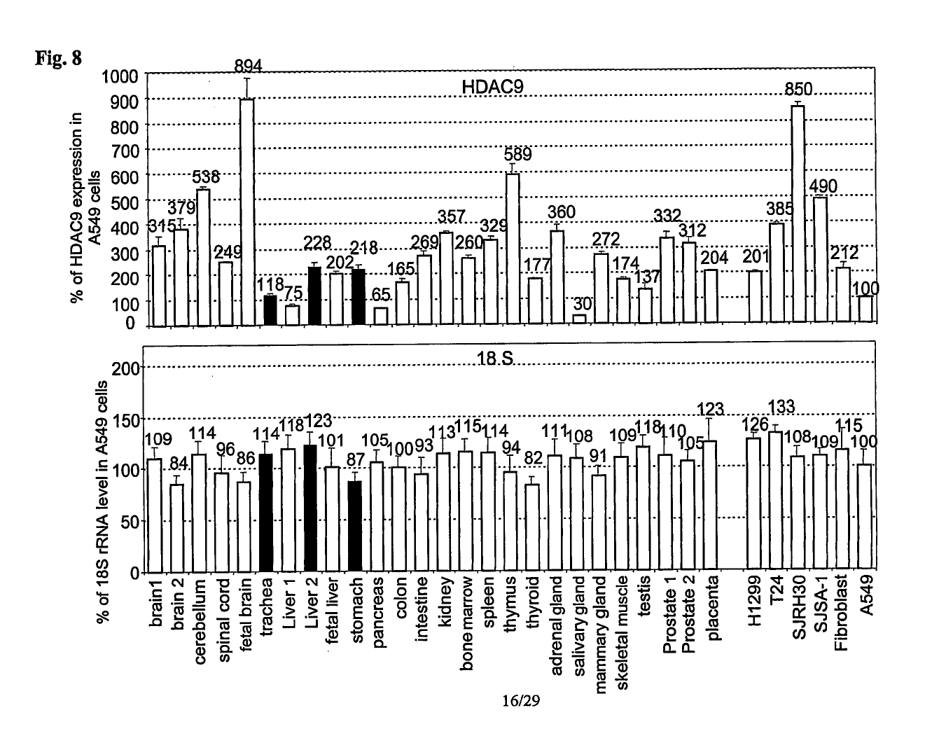

mRNA distribution of HDAC9 in normal tissues is investigated using Northern analysis. Probes are prepared by ³²P-labeling a 750 bp EcorV/Not1 HDAC9 fragment using Redi-Prime random nucleotide labelling kit according to manufacturer's instructions (Amersham, Piscataway, N.J.). A Northern blot containing polyA+RNA from 12 normal tissues (Origene Technologies, Rockville, Md.) and an array of matched tumor versus normal cDNAs (Clontech, Palo Alto, Calif.) are probed with the [³²P]-labeled 750 bp EcorV/Not1 HDAC9 fragment and washed under high stringency conditions (68° C.). Hybridized blots are washed two times for 15 min at 68° C. in 2×SSC/0.1% SDS followed by two 30 min washes in 0.1×SSC/0.1% SDS at 68° C. The blot is exposed to film with an intensifying screen for 18 hr. Results indicate that an approximately 3.0 Kb HDAC9 mRNA is detected in brain, colon, heart kidney, liver, lung, placenta, small intestine, spleen, stomach and testes. HDAC9 message was not detected in muscle, but GAPDH was also not detected. See FIG. 7.

Analogous computer techniques using BLAST (Altshul, S. F. 1993, 1990 refs) are used to search for identical or related molecules in nucleotide databases such as GenBank or the LIFESEQ™ database. The basis of the search is the product score which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1-2% error; and at 70, the match will be exact. Homologous molecules are usually identified by selecting those which show product scores between 15 and 40, although lower scores may identify related molecules.

The results of Northern analysis are reported as a list of libraries in which the transcript encoding HDAC9 occurs. Abundance and percent abundance are also reported. Abundance directly reflects the number of times a particular transcript is represented in a cDNA library, and percent abundance is abundance divided by the total number of sequences examined in the cDNA library.

In this case, electronic Northern analysis of LIFESEQ™ database (Incyte Pharmaceuticals, Inc. Palo Alto, Calif.) indicates tissue distribution of the HDAC9 sequence as seen in Table 5. These results are reported as a list of cDNA libraries in which the transcript encoding HDAC9 occurs. The presence of HDAC9 in 20 libraries from different tissue-specific and mixed tissue sources indicates that HDAC9, like other HDAC family members may be found as an expressed gene in a wide range of tissues. This result is supported by the Northern hybridization of an HDAC9 probe to mRNAs from 12 normal tissues (see FIG. 7).

TABLE 5
Tissue distribution determined electronically from LIFESEQ ™
database.

Tissue Category
Cardiovascular System
Connective Tissue
Digestive System
Embryonic Structures
Endocrine System
Exocrine Glands
Genitalia, Female
Genitalia, Male
Germ Cells
Hemic and Immune System
Liver
Musculoskeletal System
Nervous System
Pancreas
Respiratory System
Sense Organs
Skin
Stomatognathic System
Unclassified/Mixed
Urinary Tract

### Example 6

### Real Time PCR Survey of HDAC9 Distribtuion in Human Normal Tissues and Cell Lines

Real Time PCR. Total RNA from cultured cell lines was isolated with the Rneasy 96 kit according to the manufacturers protocol (Qiagen, Valencia Calif.). RNA from human tissues was purchased (Clontech Inc, Palo Alto, Calif.) and the tissue sources are listed in table 6 below.

TABLE 6
Tissue sources of RNA for real time PCR analysis

| | | Age range | Number of |
|---|---|---|---|
| | Sex of | of donor | samples |
| Tissue | donor | (yrs.) | pooled |
| Brain 1 | M | 57 | 1 |
| Brain 2 | F | 16&36 | 2 |
| Cerebellum | M | 64 | 1 |
| Spinal cord | M/F | 17-72 | 31 |
| Fetal brain | M/F | 20-23 wks | 8 |
| Trachea | M/F | 17-70 | 84 |
| Liver 1 | M | 27 | 1 |
| Liver 2 | M/F | 15&35 | 2 |
| Fetal liver | ? | 15-24 wks | ? |
| Stomach | M/F | 23-61 | 15 |
| Pancreas | M/F | 17-69 | 18 |
| Colon | M | 35&50 | 2 |
| Intestine | M/F | 25&30 | 2 |
| Kidney | M/F | 24-55 | 8 |
| Bone | M/F | 18-68 | 24 |
| marrow | | | |
| Spleen | M | 22-60 | 7 |
| Thymus | M | 6-45 | 9 |
| Thyroid | M/F | 10-46 | 4 |
| Adrenal | M | 32-50 | 6 |
| gland | | | |
| Salivary | M/F | 13-78 | 43 |
| gland | | | |
| Mammary | F | 23-47 | 8 |
| gland | | | |
| Skeletal | M/F | 23-56 | 10 |
| muscle | | | |
| Testis | M | 28-64 | 25 |
| Prostate 1 | M | 26-64 | 23 |
| Prostate 2 | M | 14-60 | 10 |
| Placenta | F | 22-41 | 15 |

Numbers following tissues represent separate samples from the same tissue type; Male (M), Female (F)

Human cell lines, H1299 human lung carcinoma, T24 bladder carcinoma, SJRH30 muscle rhabdomyosarcoma, SJSA-1 osteosarcoma, human fibroblasts, and A549 human lung carcinoma, were obtained from American Type Tissue Culture Collection. Total RNA was isolated from human cell lines using RNA easy kit according to the manufacturers instructions (Qiagen, Valencia, Calif.). RNAs were quantified using RT-PCR on an ABI Prism Sequence Detection System. The primers used for detection of HDAC9 were forward primer 5′-GGATCCAGTATCTCTT TGAGGATGAC-3′, reverse primer 5′-AGAAGCGCCCATGCTCATA-3′, and Taqman probe 5′-AGCGTCCTTTACT TCTCCTGGCACCG-3′. The Taqman Reaction System (Eurogentec, Belgium) was used with 10 ng total RNA in a 25 μl reaction in the proportions indicated by the manufacturer but supplemented with 0.25 U/μl reverse transcriptase (ultiScribe ABI, Perkin Elmer, Branchburg N.J.) and 0.08 U/μl RNaseOUT RNAse inhibitor (Life Technologies, Gaithersburg, Md.). The reverse reaction was initiated with a 5 min incubation at 48° C. for the reverse transcription of the mRNA followed by a 10 min incubation at 95° C. to inactivate the reverse transcriptase and simultaneously activate the ‘hot-start’ thermostable DNA polymerase. This was followed by 50 cycles of a two-step PCR reaction with alternating 15 sec at 95° C. and 60 sec at 60° C. Computations were performed using ABI sequence detection software (version 1.6.3). The RT-PCR assays were standardized with cRNAs transcribed in vitro with the T7 RNA polymerase reaction using the Maxiscript kit (AMBION Inc., Austin, Tex.) according to the manufacturers protocol. The RT-PCR assays were standardized with a dilution series of total RNA isolated from A549 lung tumor cells. Parallel to the RT-PCR, the total amount of RNA in each reaction was quantitated in a fluorometric assay using the RiboGreen kit (Molecular Probes Inc., address) according to the manufacturers instructions, using mammalian ribosomal RNA provided with the kit as standard.

Real time PCR was also used to survey the distribution and levels of HDAC9 in tissues and tumor cell lines, relative to the levels of 18S ribosomal RNA. RNA from the human A549 lung carcinoma cell line was arbitrarily chosen as an internal control for the levels of total RNA in the samples. The levels of HDAC9 and 18S rRNA in A549 cells were set at 100% and the levels of HDAC9 and 18S rRNA in other tissues and cell lines were measured as a percent of the level of these genes in A549 RNA. The levels of 18S nbosomal RNA ranged between 82% and 126% of the A549 internal control in all of the RNA samples, suggesting that there were similar amounts of RNA in the analyzed tissue samples. HDAC9 was detected at varying levels by real time PCR in a wide range of tissues (FIG. 8), confining the Northern blot analysis (FIG. 7). In normal tissues, HDAC9 was detected at the highest levels in fetal brain (894%), cerebellum (538%), and thymus (589%). In tumor cell lines, HDAC9 was detected at the highest levels in SJRH30 cells (850%) (FIG. 8). These results suggest that HDAC9 is differentially expressed in some tissues at the RNA level.

### Example 7

### HDAC Enzyme Assay

Preparation of HDAC9-flag. A flag epitope tag sequence was added to the 3′ end of HDAC9v1 by PCR The PCR primers were 5′-ACGCCGGATATCACATTGGT TCTGC-3′ and 5′-GCGGAATTCTTATTATTTATCATCATCATCTTTATAATCCCC GTCGACAGCCACCAGGTGAGGATGGCA-3′. The flag-tagged HDAC9v1 was reconstructed using the EcoRV site in the 1^{st} primer and subcloned into the XbaI and EcoRI sites of human expression vector pCDNA3.1(−) (Invitrogen, Carlsbad, Calif.).

HDAC activity assay. HDAC activity assays are performed as previously described (Emiliani, S., Fischle, W., Van Lint, C., Al-Abed, Y., and Verdin, E. (1998) Proc. Natl. Acad. Sci. U.S.A. 95,2795-2800). 5×10⁶ 293 cells grown to 50% confluency in 100 mm dishes are transfected with 30 ug of C-terminally flag-tagged HDAC1, HDAC3, HDAC4, HDAC6, HDAC7, or HDAC9 using Geneporter transfection kit according to the manufacturers instructions. The cell culture medium is changed 5 h after transfection. 48 h after transfection cells are washed in cold PBS and scraped into 1 ml of IP buffer (50 mM Tris-HCl pH 7.5, 120 mM NaCl, 0.5 mM EDTA, 0.5% NP-40) and incubated on a rocker for 20 min. Cellular debris is pelleted in a centrifuge at 14K for 20 min. The supernatant is precleared for 1 h with protein G beads (Pharmacia Biotech) in IP buffer. Imnunoprecipitations are performed by incubating the precleared supernatant with either α-FLAG M2 agarose affinity gel (Sigma) for 2 h at 4° C. or anti-HDAC2 (Santa Cruz) for 1 h followed by incubation with protein G beads for 1 h at 4° C. The beads are then washed three times for 5 min in IP buffer and then washed three times in high salt IP buffer (50 mM Tris-HCl pH 7.5, 1000 mM NaCl, 0.5 mM EDTA, 0.5% NP-40) at 4° C. IPS are then washed two times for 2 min in 1 ml of HD-buffer (10 mM Tris-HCl pH 8.0, 10 mM NaCl, 10% glycerol). When trapoxin inhibition is determined Ips are incubated with 0.3, 3, 30 and 300 nM TPX in HD-buffer for 20 min. Supernatants are incubated with 100000 cpm substrate ([³H]-Ac(H41-24) SGRGKGGKGLGKGGAKRHRKVLRD, in vitro/chemically acetylated using BOP-chemistry) in 30 ul HD-buffer or TPX in HD-buffer, resuspending the sepharose by gently tapping the tube and shaking in an Eppendorf 5436 Thermomixer at full speed at 37° C. for 2 h. 170 ul HD-buffer and 50 ul stop-mix (1M HCl, 0.16M HAc) are added, vortexed for 15′ min, 600 ul ethylacetate is then added and vortexed for 45 minutes, then centrifuged at 14000 g for 7 minutes. 540 ul of the organic (upper) phase is then counted in 5 ml scintillation liquid using conventional techniques.

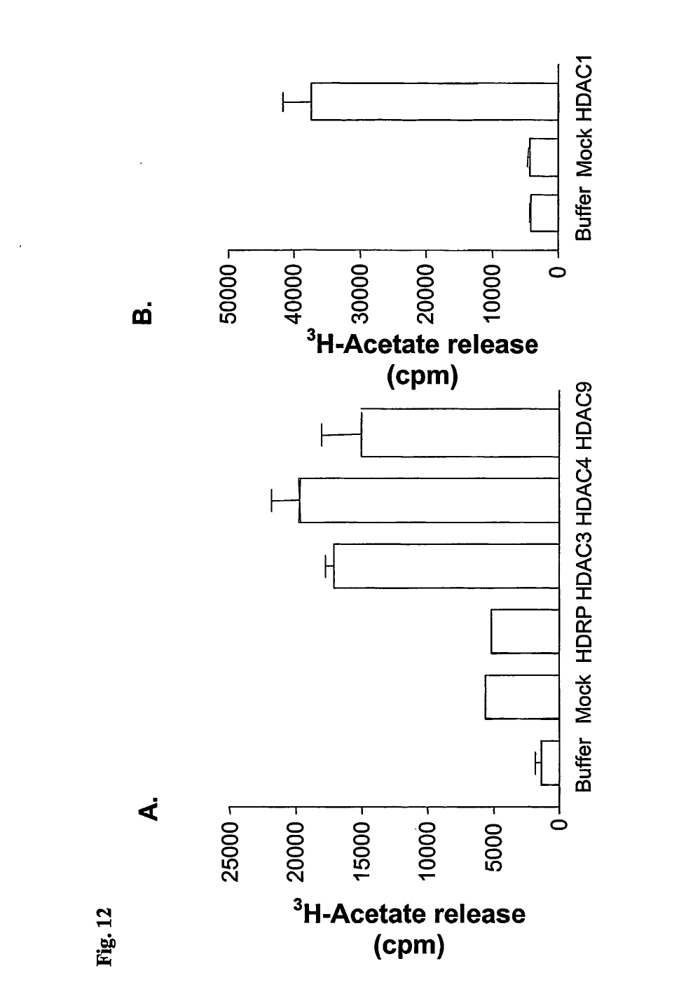

HDAC9 is catalytically active. In vitro histone deacetylase assays using immunoprecipiated HDAC9 and an ³H-acetylated histone H4 peptide as substrate were performed to determine whether HDAC9 was catalytically active and to compare the activity of HDAC9 to known catalytically active HDAC1, HDAC3, and HDAC4. An HDAC-related protein that lacks catalytic activity, HDRP/MITR/HDACC was used as a negative control (Zhou, X., Richon, V. M., Rifkind, R. A., Marks, P. A. (2000) Identification of a transcriptional repressor related to the noncatalytic domain of histone deacetylases 4 and 5. Proc Natl Acad Sci USA 97, 1056-61). These results demonstrated that HDAC9 could deacetylate the histone peptide substrate at a level that was equivalent to UDAC3 and HDAC4 (FIG. 12A), while HDAC1 was more effective in this assay (FIG. 12B).

### Example 8

### HDAC9 Expression and Cellular Localization

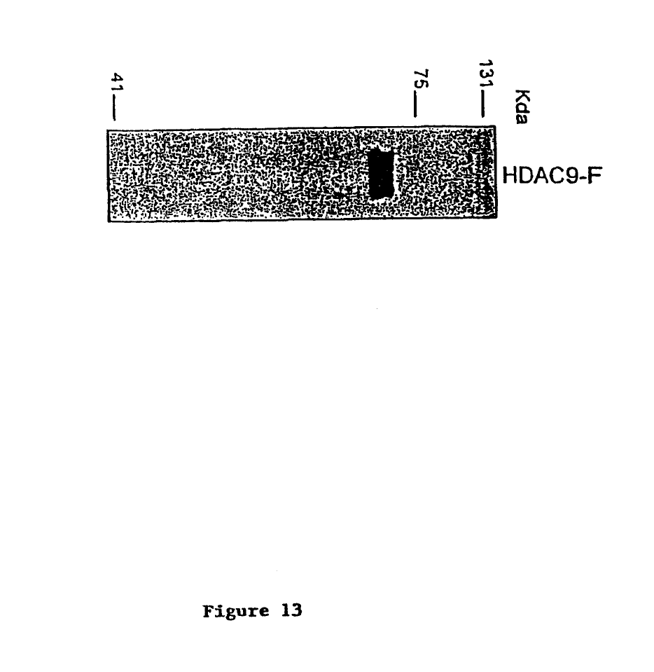

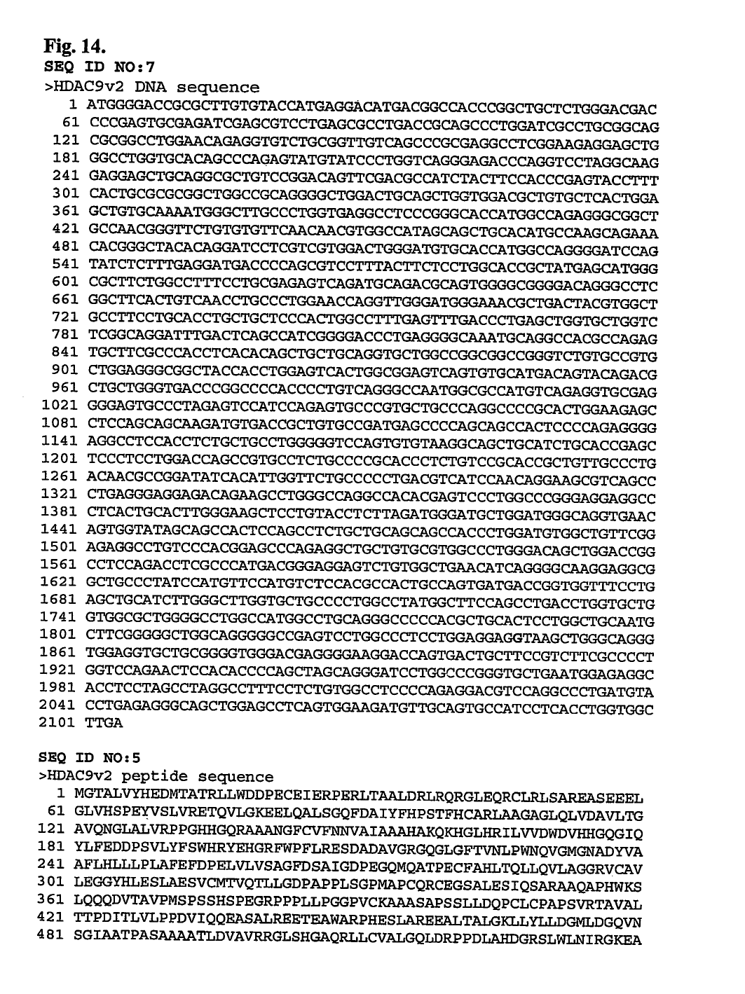

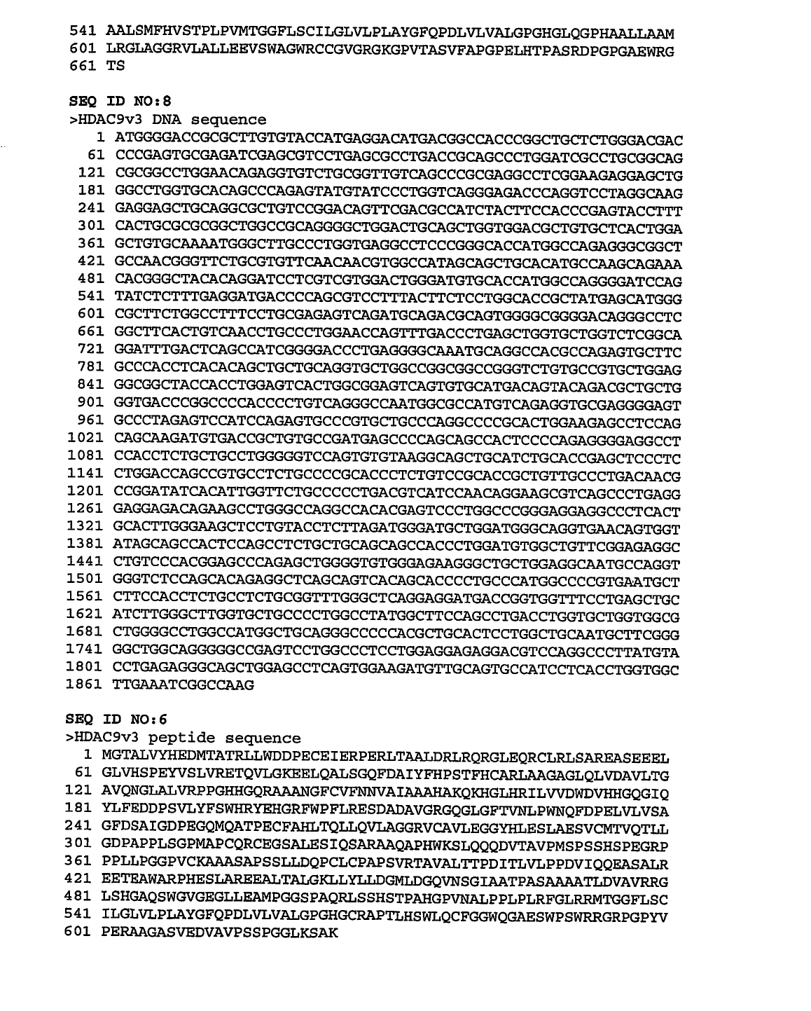

HDAC9 is expressed in vitro using 1 ug of the M6 clone, 2 ul of ³⁵S-Methionine and Sp6 TNT Quick Coupled Transcription/Translation System according to manufacturer instructions. (Promega, Madison, Wis.). Proteins are electrophoresed on a SDS-PAGE gel according to conventional methods and visualized by a Storm phosphorimager. The complete HDAC9 sequence molecular weight is estimated in silico as 72 kda using VectorNTI Suite software (Informax, North Bethesda, Md.). A doublet was observed on a 10% SDS-PAGE gel. Doublets have also been observed when HDAC1 is translated in vitro. These doublets suggest that there is potentially a second translation initiation site. Furthermore, these results suggest that HDAC9 is an expressed gene. See FIG. 13.

1×10⁵ Cos7 cells are plated onto chamber slides. Cells are transfected on the slides with 2 ug of flag epitope-tagged HDAC9 or a cytoplasmically expressed protein (Ena-flag) using Geneporter2 in serum free medium according to the manufacturers instructions. The cell culture medium is changed 24 h after transfection. 48 h after transfection, cells are washed three times with PBS, fixed for 15 min. in 5% formaldehyde, washed two times in PBS, and blocked for 30 minutes at room temperature in 10% fetal calf serum (Sigma) in PBS with 0.5% Triton-X-100 to permeablize the cells. The cells are washed again two times in PBS and then incubated with 25 mg/ml anti-Flag-FITC conjugate for 1 hour. The stained cells are washed with PBS and photographed using fluorescence microscopy.

HDAC9 is a nuclear protein. The translated HDAC9 peptide sequence predicts a 72 Kda protein and this was confirmed by in vitro translation (FIG. 13A). In order to determine the cellular localization of HDAC9, flag epitope-tagged HDAC9, Enabled (Ena) or pCMV4flag were transfected into Cos7 and 293 cells or cells were mock transfected without plasmid. The flag epitope was detected by fluorescence immunocytochemistry 48 h after transfection (FIG. 13B). Ena is a cytoskeleton-associated cytoplasmic protein substrate of Ab1 tyrosine kinase that transduces the axon-repulsive function of the Roundabout receptor during axon guidance (Gertler F B, Comer A R, Juang J L, Ahern S M, Clark M J, Liebl E C, Hoffmann F M. (1995) enabled, a dosage-sensitive suppressor of mutations in the Drosophila Ab1 tyrosine kinase, encodes an Ab1 substrate with SH3 domain-binding properties. Genes Dev. 9, 521-533.Bashaw G J, Kidd T, Murray D, Pawson T, Goodman C S. (2000) Repulsive axon guidance: Abelson and Enabled play opposing roles downstream of the roundabout receptor. Cell.101, 703-715). As expected, Ena was detected in the cytoplasm, whereas HDAC9 was detected in the nuclei of these cells. The detection of HDAC9 in the nuclei of both Cos7 and 293 cells suggested that HDAC9 was predominantly a nuclear protein.

### Example 9

### Identification of Associated Proteins in HDAC Complexes

Transfection. 1×10⁷ Cos7 cells are transfected with 10 ug of either C-terminally flag epitope-tagged HDAC1, HDAC2, HDAC3, HDAC4, HDAC6, HDAC7, or HDAC9 in pCDNA3.1 expression vector or Flag vector or buffer (Mock) as transfection controls by electroporation using a Gene Pulser II instrument (Biorad, Hercules Calif.) set at 0.3Kv/500 uF.

Immunoprecipitati n. Immunoprecipitations are performed as described (Grozinger, C. M., Hassig, C. A., and Schreiber, S. L. 1999. Proc. Natl. Acad. Sci. USA 96, 4868-4873). Whole cell extracts are prepared 48 h after transfection by scraping cells into JLB buffer (50 mM Tris-HCL, pH 8, 150 mM NaCl, 10% glycerol, 0.5% Triton-X-100) containing complete protease inhibitor cocktail (Boehringer-Mannheim). Lysis is continued at 4° C. for 10 min. and then cellular debris is pelleted by centrifugation at 14K for 5 minutes. Supernatants are pre-cleared with Sepharose A/G-plus agarose beads (Santa Cruz). Recombinant proteins are immunoprecipitated from pre-cleared supernatant by incubation with α-FLAG M2 agarose affinity gel (Sigma) for 2 h at 4° C. or anti-HDAC1 (Santa Cruz, Santa Cruz, Calif.) for 1 h at 4° C., followed by incubation with Sepharose A/G beads. For Western blot analysis, the beads are washed with MSWB buffer (50 mM Tris-HCl, pH 8, 150 mM NaCl, 1 mM EDTA, 0.1% NP-40) and the proteins are separated by SDS/PAGE. Western blots are probed with anti-flag M2 (Sigma), HDAC1 (Santa Cruz), anti-HDAC2 (Santa Cruz), anti-HDAC6 (Santa Cruz), anti-Rb (Pharmingen), or anti-mSin3A (Transduction Labs, Lexington, Ky.)

HDAC9 associates with proteins in the mSin3A complex. Class I HDACs, but not class II HDACs were previously found to be associated with the mSin3A complexes. The core HDAC1 complex consists of HDAC1, HDAC2, RbAp46, RbAp48. This core complex has been found to associate with an mSin3A complex that is involved in transcriptional repression through an Rb and E2F complex (Luo R X, Postigo A A, Dean D C.(1998) Rb interacts with histone deacetylase to repress transcription. Cell. 92, 463-473; Magnaghi-Jaulin L, Groisman R, Naguibneva I, Robin P, Lorain S, Le Villain J P, Troalen F, Trouche D, Harel-Bellan A. (1998) Retinoblastoma protein represses transcription by recruiting a histone deacetylase. Nature. 391, 601-605; Brehm A, Miska E A, McCance D J, Reid J L, Bannister A J, Kouzarides T. (1998) Retinoblastoma protein recruits histone deacetylase to repress transcription. Nature. 391, 597-601). In order to determine whether HDAC9 was a part of this complex, endogenous HDAC1, HDAC2, Rb, and mSin3 proteins were co-immunoprecipitated from cells transfected with flag-epitope tagged HDAC1, HDAC3, HDAC4, HDAC6, HDAC7 or HDAC9. To assure that transfected flag epitope-tagged HDACs could be detected in cells, the levels of HDAC expression were detected by immunoprecipitation and Western blotting with antiserum to the flag epitope. To determine which HDACs associated with components of the Sin3 complex, endogenous proteins in the Sin3 complex were immunoprecipitated and the associated HDACs were detected by Western blotting flag epitope-specific antibody HDAC9 was found to associate with HDAC1, HDAC2., Rb, and mSin3A, suggesting that HDAC9 is a component of an mSin3A complex.

HDAC9 associates with SMRT and NCoR. Since corepressors SMRT and NCoR associate with the mSin3 core complex, experiments were performed to co-immunoprecipitate HDACs with NCoR and SMRT (FIG. 15). HDAC9 co-immunoprecipitated with both of these proteins, suggesting that HDAC9 associates with SMRT, and NCoR. Western analysis of the flag-detected blots with anti-NCoR indicated that NCoR was immunoprecipitated. As previously reported, SMRT co-immunoprecipitated with HDAC4 and HDAC6, and HDAC6 and HDAC7 did not associate with the Sin3A complex.

HDAC9 associates with 14-3-3 and Erk proteins. HDAC4 was previously found to associate with 14-3-3-β, 14-3-3-ε, CamK, Erk1, and Erk 2 proteins, which sequester HDAC4 in the cytoplasm and prevent phosphorylated HDAC4 and HDAC5 from entering the nucleus and repressing MEF2 activated transcription. In order to determine whether HDAC9 associate with these proteins, experiments were performed to co-immunoprecipitate HDACs with 14-3-3 and Erk proteins. All of the HDACs tested associated with 14-3-3s and Erks. These results suggest that the association of HDACs with 14-3-3 and Erks might be a general mechanism of sequestering HDACs in the cytoplasm.

Classification of HDAC9. HDACs have been classified by sequence similarity to yeast HDACs, sequence length, location of catalytic domain, cellular localization, associating proteins, and sensitivity to HDAC inhibitors. The data in this study suggests that HDAC9 has characteristics of both class I and class II HDACs. HDAC9 had sequence similarity with class II yeast hda1 subfamily member Clr3 and HDAC6 catalytic domain 1. In addition, the 3 Kb HDAC9 transcript was only detected in kidney and testis, suggesting that it might have a limited tissue distribution like class II HDACs. HDAC9 was between class I and class II HDACs in length. Class I HDACs average 443 bp in length, whereas class II HDACs average 1069 bp in length. However, HDAC9 was found to have an N-terminal catalytic domain, as opposed to the C-terminal domains that have been found in class II HDACs. HDAC6 is an exception that has both N-terminal and C-terminal catalytic domains. Furthermore, class I HDACs are nuclear proteins, while class II HDACs are nucelo-cytoplasmic. Immunocytochemistry indicated that HDAC9 was predominantly nuclear and was detected in a different subcellular compartment in comparison to the Ena protein that is expressed in the cyotplasm. In contrast to the 3 Kb HDAC9 transcript that might be differentially expressed, a 3.5 Kb HDAC9 transcript that was identified by Northern analysis was expressed ubiquitously in normal tissues, tumor tissues and cell lines, similar to class I HDACs. In addition, HDAC9 was found to co-immunoprecipitate with proteins that were previously only associated with class I HDAC complexes, including HDAC1, HDAC2, mSin3A, and Rb. HDAC9 also has putative C-terminal LXCXE motifs that so far have only been found in HDAC1. HDAC9 was also found to associate with NCoR and SMRT. This evidence suggests HDAC9 had characteristics that bridged those of class I and class II HDACs.

What is claimed is:

1. An isolated polypeptide comprising the amino acid sequence set forth in SEQ ID NO:1, SEQ ID NO 5 or SEQ ID NO 6.

2. An isolated polypeptide consisting of the amino acid sequence set forth in SEQ ID NO:1, SEQ ID NO 5 or SEQ ID NO 6.

3. An isolated DNA comprising a nucleic acid sequence that encodes the polypeptide of claim 1 or 2.

4. A vector molecule comprising at least a fragment of the isolated DNA according to claim 3.

5. The vector molecule according to claim 4 comprising transcriptional control sequences.

6. A host cell comprising the vector molecule according to claim 5.

7. The isolated DNA according to claim 3, comprising a nucleotide sequence selected from the group consisting of (1) the nucleotide sequence set forth in SEQ ID NO:2, 7 or 8, being the complete cDNA sequence encoding the polypeptide as defined in claim 2; (2) the nucleotide sequence set forth in SEQ ID NO:3, being the open reading frame of the cDNA sequence encoding the polypypetide as defined in claim 2; (3) a nucleotide sequence capable of hybridizing under high stringency conditions to a nucleotide sequence set forth in SEQ ID NO:3; and (4) the nucleotide sequence set forth in SEQ ID NO:4, being the endogenous genomic human DNA encoding the polypeptide as defined in claim 2.

8. A vector molecule comprising at least a fragment of an isolated DNA molecule according to claim 7.

9. The vector molecule according to claim 8 comprising transcriptional control sequences.

10. A host cell comprising the vector molecule according to claim 9.

11. A host cell which can be propagated in vitro and which is capable upon growth in culture of producing a polypeptide according to claim 1 or 2, wherein said cell comprises at least one transcriptional control sequence that is not a transcriptional control sequence of the natural endogeneous human gene encoding the polypeptide of claim 2, wherein said one or more transcriptional control sequences control transcription of a DNA encoding a polypeptide according to claim 1 or 2.

12. A method for the diagnosis of a condition associated with abnormal regulation of gene expression which includes, abnormal cell proliferation, cancer, atherosclerosis, inflammatory bowel disease, host inflammatory or immune response, or psoriasis in a human which comprises: detecting abnormal transcription of messenger RNA transcribed from the natural endogeneous human gene encoding the polypeptide as defined in claim 2 in an appropriate tissue or cell from a human, wherein said abnormal transcription is diagnostic of said condition.

13. The method of claim 12, wherein said natural endogeneous human gene comprises the nucleotide sequence set forth in SEQ ID NO:4, 7 or 8.

14. The method of claim 12, comprising contacting a sample of said appropriate tissue or cell or contacting an isolated RNA or DNA molecule derived from said tissue or cell with an isolated nucleotide sequence of at least about 15-20 nucleotides in length that hybridizes under high stringency conditions with the isolated nucleotide sequence as defined in claim 3.

15. A method for the diagnosis of a condition associated with abnormal HDAC9 expression or activity in a human which comprises:
measuring the amount of a polypeptide comprising the amino acid sequence set forth in SEQ ID NO:1, 5 or 6 or fragments thereof, in an appropriate tissue or cell from a human suffering from said condition wherein the presence of an abnormal amount of said polypeptide or fragments thereof, relative to the amount of said polypeptide or fragments thereof in the respective tissue from a human not suffering from said condition associated with abnormal HDAC9 expression or activity is diagnostic of said human's suffering from a condition

16. The method of claim 15, wherein said detecting step comprises contacting said appropriate tissue or cell with an antibody which specifically binds to a polypeptide that comprises the amino acid sequence set forth in SEQ ID NO:1, 5 or 6 or a fragment thereof and detecting specific binding of said antibody with a polypeptide in said appropriate tissue or cell, wherein detection of specific binding to a polypeptide indicates the presence of a polypeptide that comprises the amino acid sequence set forth in SEQ ID NO:1, 5 or 6 or a fragment thereof.

17. An antibody or a fragment thereof which specifically binds to a polypeptide that comprises the amino acid sequence set forth in SEQ ID NO:1, 5 or 6 or to a fragment of said polypeptides.

18. An antibody fragment according to claim 17 which is an Fab or F(ab′)₂ fragment.

19. An antibody according to claim 17 which is a polyclonal antibody.

20. An antibody according to claim 17 which is a monoclonal antibody.

21. A method for producing a polypeptide as defined in claim 1 or 2, which method comprises:
culturing a host cell having incorporated therein an expression vector comprising an exogenously-derived polynucleotide encoding a polypeptide comprising an amino acid sequence as set forth in SEQ ID NO:1, 5 or 6 under conditions sufficient for expression of the polypeptide in the host cell, thereby causing the production of the expressed polypeptide.

22. The method according to claim 21, said method further comprising recovering the polypeptide produced by said cell.

23. The method according to claim 21, wherein said exogenously-derived polynucleotide encodes a polypeptide consisting of an amino acid sequence set forth in SEQ ID NO:1, 5 or 6.

24. The method according to claim 21, wherein said exogenously-derived polynucleotide comprises the nucleotide sequence as set forth in SEQ ID NO:2, 7 or 8.

25. The method according to claim 21, wherein said exogenously-derived polynucleotide comprises the nucleotide sequence as set forth in SEQ ID NO:3.

26. The method accoding to claim 21, wherein said exogenously-derived polynucleotide consists of the nucleotide sequence as set forth in SEQ ID NO:3.

27. The method according to claim 24, wherein said exogenously-derived polynucleotide comprises the nucleotide sequence as set forth in SEQ ID NO:4.
